SEQUENCE LISTING (61 sequences)

SEQ ID NO: 1 - protein, 248 aa, synthetic construct
  source 1..248: note = scFv from the mouse scFv library
DYKDIVMTQSHKFMSTSVGDRVSITCKASQDVGTAVAWYQQKPGQSPKLLIYWASTRHTGVPDRFTGSGSGTDFTLTISNVQSEDLADYFCQQYSSYLTFGAGTKLELKRGGGGSGGGGSGGGGSGGGGSEVMLVESGGDLVKPGGSLKLSCAASGFTFSSYGMSWVRQTPDKRLEWVATISSGGSYTYYPDSVKGRFTISRDNAKNTLYLQMSSLKSEDTAMYYCALLRSDWYFDVWGAGTTVTVSS

SEQ ID NO: 2 - protein, 12 aa, synthetic construct
  source 1..12: note = CDRH3 of the scFv of SEQ ID NO:1
ALLRSDWYFDVW

SEQ ID NO: 3 - protein, 330 aa, synthetic construct [sequence omitted]

SEQ ID NO: 4 - protein, 340 aa, synthetic construct
  source 1..340: note = Grafted protein comprising a WT hIgG1 constant sequence comprising a heterologous CDRH3 insertion in the CH3 domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESMLLRSDWYFDVWQPENNYKTTPPVLDSDGSFFLYSKLTVDKSRWQQGNVFSCSVMHEALHNHYTQKSLSLSPGK

SEQ ID NO: 5 - protein, 348 aa, synthetic construct
  source 1..348: note = GLP1.1 - modified version of LP1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGGCMLLRSDWYFDVWCGGGQPENNYKTTPPVLDSDGSFFLYSKLTVDKSRWQQGNVFSCSVMHEALHNHYTQKSLSLSPGK

SEQ ID NO: 6 - protein, 348 aa, synthetic construct
  source 1..348: note = GLP1.2 - modified version of LP1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGGGMLLRSDWYFDVWGGGGQPENNYKTTPPVLDSDGSFFLYSKLTVDKSRWQQGNVFSCSVMHEALHNHYTQKSLSLSPGK

SEQ ID NO: 7 - protein, 348 aa, synthetic construct
  source 1..348: note = BLP1.1 - modified version of LP1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGCMLLRSDWYFDVWCGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 348 aa, synthetic construct
  source 1..348: note = BLP1.2 - modified version of LP1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELGGGMLLRSDWYFDVWGGGTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 760 aa, synthetic construct
  source 1..760: note = Human transferrin receptor protein 1
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 10 - protein, 8 aa, synthetic construct
  source 1..8: note = Heterologous insertion
LRYGRQYS

SEQ ID NO: 11 - protein, 14 aa, synthetic construct
  source 1..14: note = Heterologous insertion
NVYAMTRNWWSDVY

SEQ ID NO: 12 - protein, 16 aa, synthetic construct
  source 1..16: note = Heterologous insertion
NVWHDLVWSVSTTDVY

SEQ ID NO: 13 - protein, 17 aa, synthetic construct
  source 1..17: note = Heterologous insertion
NVYARPRPDNLNWSDVY

SEQ ID NO: 14 - protein, 17 aa, synthetic construct
  source 1..17: note = Heterologous insertion
HVETPYDSPELNWWDVY

SEQ ID NO: 15 - protein, 19 aa, synthetic construct
  source 1..19: note = Heterologous insertion
NVYELVEDTSAYEIGVDVY

SEQ ID NO: 16 - protein, 8 aa, synthetic construct
  source 1..8: note = Heterologous insertion
TGWGFDYW

SEQ ID NO: 17 - protein, 10 aa, synthetic construct
  source 1..10: note = Heterologous insertion
TRGATALDYW

SEQ ID NO: 18 - protein, 14 aa, synthetic construct
  source 1..14: note = Heterologous insertion
NVYAMTRNWWSDVY

SEQ ID NO: 19 - protein, 16 aa, synthetic construct
  source 1..16: note = Heterologous insertion
NVWHDLVWSVSTTDVY

SEQ ID NO: 20 - protein, 17 aa, synthetic construct
  source 1..17: note = Heterologous insertion
NVYARPRPDNLNWSDVY

SEQ ID NO: 21 - protein, 17 aa, synthetic construct
  source 1..17: note = Heterologous insertion
HVETPYDSPELNWWDVY

SEQ ID NO: 22 - protein, 19 aa, synthetic construct
  source 1..19: note = Heterologous insertion
NVYELVEDTSAYEIGVDVY

SEQ ID NO: 23 - protein, 9 aa, synthetic construct
  source 1..9: note = Heterologous insertion
ARGLSGNYV

SEQ ID NO: 24 - protein, 9 aa, synthetic construct
  source 1..9: note = Heterologous insertion
ARGLSGNFV

SEQ ID NO: 25 - protein, 10 aa, synthetic construct
  source 1..10: note = Heterologous insertion
GLSGNYVMDY

SEQ ID NO: 26 - protein, 10 aa, synthetic construct
  source 1..10: note = Heterologous insertion
GLSGNYVVDY

SEQ ID NO: 27 - protein, 10 aa, synthetic construct
  source 1..10: note = Heterologous insertion
GLSGNFVMDF

SEQ ID NO: 28 - protein, 6 aa, synthetic construct
  source 1..6: note = Heterologous insertion
ARGTRA

SEQ ID NO: 29 - protein, 7 aa, synthetic construct
  source 1..7: note = Heterologous insertion
GTRAYHY

SEQ ID NO: 30 - protein, 8 aa, synthetic construct
  source 1..8: note = Heterologous insertion
GTYRAYHF

SEQ ID NO: 31 - protein, 12 aa, synthetic construct
  source 1..12: note = Heterologous insertion
ARYGYGNPATRY

SEQ ID NO: 32 - protein, 13 aa, synthetic construct
  source 1..13: note = Heterologous insertion
YGYGNPATRYFDV

SEQ ID NO: 33 - protein, 11 aa, synthetic construct
  source 1..11: note = Heterologous insertion
TRGGYDSRAWF

SEQ ID NO: 34 - protein, 11 aa, synthetic construct
  source 1..11: note = Heterologous insertion
GGYDSRAWFAY

SEQ ID NO: 35 - protein, 11 aa, synthetic construct
  source 1..11: note = Heterologous insertion
GGYDSRAWFAH

SEQ ID NO: 36 - protein, 14 aa, synthetic construct
  source 1..14: note = Heterologous insertion
ARQGALYDGYYRGA

SEQ ID NO: 37 - protein, 15 aa, synthetic construct
  source 1..15: note = Heterologous insertion
QGALYDGYYRGAMDY

SEQ ID NO: 38 - protein, 11 aa, synthetic construct
  source 1..11: note = Heterologous insertion
ARRGYGYDGEF

SEQ ID NO: 39 - protein, 12 aa, synthetic construct
  source 1..12: note = Heterologous insertion
RGGYGYDGEFAY

SEQ ID NO: 40 - protein, 10 aa, synthetic construct
  source 1..10: note = Heterologous insertion
  VARIANT 6: note = Wherein X can be Y or F
  VARIANT 8: note = Wherein X can be M or V
  VARIANT 10: note = Wherein X can be Y or F
GLSGNXVXDX

SEQ ID NO: 41 - protein, 7 aa, synthetic construct
  source 1..7: note = Heterologous insertion
  VARIANT 7: note = Wherein X can be Y or F
GTRAYHX

SEQ ID NO: 42 - protein, 15 aa, synthetic construct
  source 1..15: note = Heterologous insertion
  VARIANT 1: note = Wherein X can be G or Q or R
  VARIANT 3: note = Wherein X can be Y or A or G
  VARIANT 4: note = Wherein X can be D or L or Y
  VARIANT 5: note = Wherein X can be S or Y or G
  VARIANT 6: note = Wherein X can be R or D or Y
  VARIANT 7: note = Wherein X can be A or G or D
  VARIANT 8: note = Wherein X can be W or Y or G
  VARIANT 9: note = Wherein X can be F or Y or E
  VARIANT 10: note = Wherein X can be R or F or the absence of a residue
  VARIANT 11: note = Can be G or the absence of a residue
  VARIANT 12: note = Can be A or the absence of a residue
  VARIANT 13: note = Can be M or the absence of a residue
  VARIANT 14: note = Wherein X can be A or D
  VARIANT 15: note = Wherein X can be Y or H
XGXXXXXXXXGAMXX

SEQ ID NO: 43 - protein, 9 aa, synthetic construct
  source 1..9: note = Heterologous insertion
VRLRGYFDY

SEQ ID NO: 44 - protein, 16 aa, synthetic construct
  source 1..16: note = Heterologous insertion
ARRDGSYYPYYWYFDL

SEQ ID NO: 45 - protein, 17 aa, synthetic construct
  source 1..17: note = Heterologous insertion
ARTLINYSDYADYVMDA

SEQ ID NO: 46 - protein, 17 aa, synthetic construct
  source 1..17: note = Heterologous insertion
ARSLINYRNYGDYVMDA

SEQ ID NO: 47 - protein, 14 aa, synthetic construct
  source 1..14: note = Heterologous insertion
ARNGVYHNYWYFDF

SEQ ID NO: 48 - protein, 15 aa, synthetic construct
  source 1..15: note = Heterologous insertion
AKEGGWFLRIYGMDY

SEQ ID NO: 49 - protein, 119 aa, synthetic construct
  source 1..119: note = Exemplary CNS antigen binding VH - BACE1
EVQLVESGGGLVQPGGSLRLSCAASGFTFSGYAIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 50 - protein, 108 aa, synthetic construct
  source 1..108: note = Exemplary CNS antigen binding VL - BACE1
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 51 - protein, 126 aa, synthetic construct
  source 1..126: note = Exemplary CNS antigen binding VH - BACE1
EVQLVESGGGLVQPGGSLRLSCAASGFNFYYSSIHWVRQAPGKGLEWVASISPYSGYTSYADSVKGRFTISADTSKNTAYLQMNSLRAEDTAVYYCARQPTHYYYYAKGYKAMDYWGQGTLVTVSS

SEQ ID NO: 52 - protein, 108 aa, synthetic construct
  source 1..108: note = Exemplary CNS antigen binding VL - BACE1
DIQMTQSPSSLSASVGDRVTITCRASQSVSSAVAWYQQKPGKAPKLLIYSASSLYSGVPSRFSGSGSGTDFTLTISSLQPEDFATYYCQQYSYSPFTFGQGTKVEIKR

SEQ ID NO: 53 - protein, 116 aa, synthetic construct
  source 1..116: note = Exemplary CNS antigen binding VH - Alpha-synuclein
EVQLLESGGGLVQPGGSLRLSCAASGFTFSRFTMTWVRQAPGKGLEWVSAISGSGGGTSYADSVKGRLTVSRDNSKNTLYLQMNSLRAEDTAVYYCAKNWAPFDYWGQGTLVTVSS

SEQ ID NO: 54 - protein, 108 aa, synthetic construct
  source 1..108: note = Exemplary CNS antigen binding VL - Alpha-synuclein
EIVLTQSPGTLSLSPGERATLSCRASQSVSRSYLAWYQQKPGQAPRLLIYGASSRATGIPDRFSGSGSGTDFTLTVSRLEPEDFAVYYCQQYGSSPWTFGQGTKVEIK

SEQ ID NO: 55 - protein, 116 aa, synthetic construct
  source 1..116: note = Exemplary CNS antigen binding VH - Alpha-synuclein
EVQLLESGGGLVQTGGSLRLSCAASGFTFSSYAMTWVRQAPGKGLEWVSAIRSNGDRTDYADSVKGRFTISRDNSQNTLYLQMNSLRAEDTAVYYCAKNWAPFDSWGQGTLVTVSS

SEQ ID NO: 56 - protein, 108 aa, synthetic construct
  source 1..108: note = Exemplary CNS antigen binding VL - Alpha-synuclein
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 57 - protein, 115 aa, synthetic construct
  source 1..115: note = Exemplary CNS antigen binding VH - Abeta
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 58 - protein, 112 aa, synthetic construct
  source 1..112: note = Exemplary CNS antigen binding VL - Abeta
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 59 - protein, 121 aa, synthetic construct
  source 1..121: note = Exemplary CNS antigen binding VH - Tau
EVQLVESGGGLVQPGGSLKLSCAASGFNFNISAIHWVRQASGKGLEWVGRIRSKSHNYATLYAASLKGRFTLSRDDSRNTAYLQMSSLQTEDMAVYYCTVLSANYDTFDYWGQGTLVTVSS

SEQ ID NO: 60 - protein, 107 aa, synthetic construct
  source 1..107: note = Exemplary CNS antigen binding VL - Tau
SYELTQPPSVSVSPGQTARISCFGDTLPKQYTYWYQQKPGQAPVLVIYKDTERPSGIPERFSGSSSGTTVTLTISGVQAEDEADYYCLSADNSATWVFGGGTKVTVL

SEQ ID NO: 61 - protein, 12 aa, synthetic construct
  source 1..12: note = Heterologous insertion
MLLRSDWYFDVW

(19) United States
(12) Patent Application Publication
(10) Pub. No.: US 2024/0383997 A1
(43) Pub. Date: Nov. 21, 2024

(54) PROTEINS COMPRISING BLOOD BRAIN BARRIER (BBB)-BINDING DOMAINS WITHIN CONSTANT DOMAINS

(71) Applicant: ALECTOR LLC, South San Francisco, CA (US)

(72) Inventors: Eric BROWN, South San Francisco, CA (US); Hamid SALIMI, San Mateo, CA (US); Wei-Hsien HO, Belmont, CA (US); Leonard PRESTA, South San Francisco, CA (US); Margaret TANG, Mountain View, CA (US); Jeonghoon SUN, San Francisco, CA (US)

(21) Appl. No.: 18/621,575

(22) Filed: Mar. 29, 2024

Related U.S. Application Data
(63) Continuation of application No. PCT/US2022/079866, filed on Nov. 15, 2022.
(60) Provisional application No. 63/279,325, filed on Nov. 15, 2021.

(51) Int. Cl. C07K 16/28; A61K 49/00; C07K 16/18; G01N 33/50

(52) U.S. Cl. CPC C07K 16/2881; A61K 49/0002; C07K 16/18; C07K 16/28; C07K 16/2803; C07K 16/2863; C07K 16/2896; G01N 33/5023; G01N 33/5064; C07K 2319/30

(57) ABSTRACT

Provided herein are engineered constant domains that have been engineered to comprise a blood brain barrier (BBB)-binding region, such a CDRH3 from an antibody that binds to a blood brain barrier target. Such engineered constant domains can be within a full length antibody or an antigen-binding fragment of an antibody such that the antibody or fragment is capable of binding to both the antigen of the antibody or fragment and the BBB target and is capable of being transported across the BBB.

### CROSS-REFERENCE TO RELATED APPLICATIONS

This application claims the benefit of U.S. Provisional Application No. 63/279,325, filed Nov. 15, 2021, which is hereby incorporated by reference herein in its entirety.

### REFERENCE TO A SEQUENCE LISTING SUBMITTED ELECTRONICALLY

The content of the electronically submitted sequence listing (Name: 4503_016PC01_Seqlisting_ST26.xml; Size: 76,961 bytes; Date of Creation: Nov. 14, 2022) is herein incorporated by reference in its entirety.

### BACKGROUND

Passive transfer of substances from blood to brain is restricted by the blood brain barrier (BBB). The BBB provides precise control of central nervous system (CNS) homeostasis allowing for proper neuronal function and also protecting neural tissue from toxins and pathogens. Alterations of the BBB are an important component of pathology and progression of different neurological diseases. However, the BBB poses a problem with regard to delivering therapeutics to the CNS. While recombinant proteins and antibody therapeutics have shown much success outside the CNS, such biologics do not cross the BBB efficiently. As a result, delivery of some therapeutics to the CNS has relied on injection of the therapeutic directly into the CNS. However, such injections are invasive procedures that have efficacy that is limited by the rapid export of cerebral spinal fluid (CSF) containing the therapeutic from the brain to the blood. Alternatively, a therapeutic intended for the CNS may be administered systemically at a high dose to allow for sufficient penetration of the BBB by the therapeutic. However, this approach may result in unintended effects due to the high dose in the periphery or increased manufacturing and formulation burdens to achieve the high dose. Accordingly, improved methods for delivering therapeutics across the BBB are needed.

### BRIEF SUMMARY OF THE INVENTION

Provided herein are polypeptides comprising an engineered constant domain, wherein the engineered constant domain comprises a heterologous insertion in a constant domain, and wherein the heterologous insertion comprises a heavy chain complementary determining region 3 (CDRH3) or a target-binding fragment thereof. In some aspects, the constant domain comprises a CH3 domain. In some aspects, the constant domain comprises a CH2 domain, a CH1 domain, or a CL domain. In some aspects, the polypeptide comprises an Fc region. In some aspects, the constant domain is a human constant domain. In some aspects,

The constant domain is a murine, rat, rabbit, or monkey constant domain. In some aspects, the CDRH3 or the target-binding fragment thereof specifically binds to a blood brain barrier (BBB) target. In some aspects, the BBB target is a BBB protein, optionally wherein the BBB protein is a BBB receptor. In some aspects, the BBB target is a BBB lipid or a BBB sugar.

In some aspects, the CDRH3 is a Kabat-defined, a Chothia-defined, an IMGT-defined, or an AbM-defined CDRH3.

Provided herein are fusion proteins comprising an engineered constant domain and a heterologous polypeptide, wherein the engineered constant domain comprises a heterologous insertion in a constant domain, and wherein the heterologous insertion comprises a CDRH3 or a target-binding fragment thereof. In some aspects, the constant domain comprises a CH3 domain. In some aspects, the constant domain comprises a CH2 domain, a CH1 domain, or a CL domain. In some aspects, the fusion protein comprises an Fc region. In some aspects, the constant domain is a human constant domain. In some aspects, the constant domain is a murine, rat, rabbit, or monkey constant domain. In some aspects, the CDRH3 or target-binding fragment thereof specifically binds to a BBB target. In some aspects, the BBB target is a BBB protein, optionally wherein the BBB protein is a BBB barrier receptor. In some aspects, the BBB target is a BBB lipid or a BBB sugar. In some aspects, the CDRH3 is a Kabat-defined, a Chothia-defined, an IMGT-defined, or an AbM-defined CDRH3.

In some aspects, the engineered constant domain is N-terminal to the heterologous polypeptide. In some aspects, the engineered constant domain is C-terminal to the heterologous polypeptide. In some aspects, the fusion protein comprises a first linker at the N-terminus of the heterologous insertion and/or a second linker at the C-terminus of the heterologous insertion. In some aspects, the first linker and the second linker have the same sequence. In some aspects, the first linker and the second linker have different sequences.

In some aspects, the heterologous polypeptide comprises an antibody or antigen-binding fragment thereof, a growth factor, a decoy receptor, or an enzyme or a catalytically active fragment thereof. In some aspects, the fusion protein does not comprise an antibody heavy chain variable region and/or an antibody light chain variable region. In some aspects, the fusion protein does not comprise an antigen-binding domain of an antibody. In some aspects, the fusion protein comprises two constant domains, optionally wherein each constant domain is an engineered constant domain comprising a heterologous insertion in a constant domain, wherein the heterologous insertion comprises a CDRH3 or a target-binding fragment thereof.

Provided herein are antigen-binding proteins comprising an engineered constant domain and an antigen-binding domain, wherein the engineered constant domain comprises a heterologous insertion in a constant domain, and wherein the heterologous insertion comprises a CDRH3 or a target-binding a fragment thereof. In some aspects, the CDRH3 or target-binding fragment thereof specifically binds to a BBB target. In some aspects, the CDRH3 is a Kabat-defined, a Chothia-defined, an IMGT—the CDRH3 or target-binding fragment thereof binds to a different target than the antigen-binding domain.

Provided herein are antigen-binding proteins comprising an engineered constant domain and an antigen-binding domain, wherein the engineered constant domain comprises a heterologous insertion in a constant domain, wherein the heterologous insertion specifically binds to a blood brain barrier target, and wherein the antigen-binding domain binds to a different target than the heterologous insertion. In some aspects, the constant domain comprises a CH3 domain. In some aspects, the constant domain comprises a CH2 domain, a CH1 domain, or a CL domain. In some aspects, the antigen-binding protein comprises an Fc region. In some aspects, the constant domain is a human constant domain. In some aspects, the constant domain is a murine, rat, rabbit, or monkey constant domain. In some aspects, the BBB target is a BBB protein, optionally wherein the BBB protein is a BBB receptor. In some aspects, the BBB target is a BBB lipid or a BBB sugar.

In some aspects, the antigen-binding protein comprises two constant domains, optionally wherein each constant domain is an engineered constant domain comprising a heterologous insertion in a constant domain, wherein the heterologous insertion comprises a CDRH3 or a target-binding fragment thereof.

In some aspects of the polypeptides, fusion proteins, and/or antigen-binding proteins provided herein, the constant domain is an IgG constant domain. In some aspects, the constant domain is an IgG1 constant domain. In some aspects, the constant domain is an IgG4 constant domain.

In some aspects of the polypeptides, fusion proteins, and/or antigen-binding proteins provided herein, the heterologous insertion is between N384 and G385 according to EU numbering or wherein the heterologous insertion replaces N384 and G385 according to EU numbering. In some aspects, the heterologous insertion is between L358 and T359 according to EU numbering or wherein the heterologous insertion replaces L358 and T359 according to EU numbering. In some aspects, the heterologous insertion is in a loop connecting beta strands A-B and E-F of a human IgG1 CH3 domain.

In some aspects of the polypeptides, fusion proteins, and/or antigen-binding proteins provided herein, the heterologous insertion comprises a CDRH3.

In some aspects of the polypeptides, fusion proteins, and/or antigen-binding proteins provided herein, the heterologous insertion comprises 5-15 amino acids or 10-15 amino acids. In some aspects, the heterologous insertion comprises 8-14 amino acids.

In some aspects of the polypeptides, fusion proteins, and/or antigen-binding proteins provided herein, the heterologous insertion binds to transferrin receptor, Basigin, Glucose Transporter Type 1 (Glut1), CD98, MfsD2a, LRP1, LRP8, or IGF1R. In some aspects, the heterologous insertion binds to an extracellular domain of transferrin receptor, Basigin, Glucose Transporter Type 1 (Glutl), CD98, MfsD2a, LRP1, LRP8, or IGF1R. In some aspects, the heterologous insertion binds to human transferrin receptor and cynomolgus transferrin receptor. In some aspects, the heterologous insertion binds to human transferrin receptor and murine transferrin receptor. In some aspects, the heterologous insertion binds to the apical domain of the transferrin receptor. In some aspects, the heterologous insertion binds to the helical domain of the transferrin receptor. In some aspects, the heterologous insertion binds to the protease-like domain of the transferrin receptor. In some aspects, binding of the polypeptide, fusion protein, or antigen-binding protein to the transferrin receptor does not block iron transport. In some aspects, binding of the polypeptide, fusion protein, or antigen-binding protein to the transferrin receptor does not decrease iron transport by more than 50%, does not decrease iron transport by more than 25%, does not decrease iron transport by more than 10%, or does not decrease iron transport by more than 5%. In some aspects, binding of the polypeptide, fusion protein, or antigen-binding protein to the transferrin receptor does not reduce the level of TfR expressing peripheral cells by more than 15% or does not reduce the level of TfR-expressing peripheral cells more than 5%. In some aspects, the peripheral cells are reticulocytes. In some aspects, the peripheral cells are lung, liver, or kidney endothelial cells. In some aspects, the polypeptide, fusion protein, or antigen-binding protein binds to a blood brain barrier target with an affinity of 1-1000 nM.

In some aspects of the polypeptides, fusion proteins, and/or antigen-binding proteins provided herein, the heterologous insertion comprises the amino acid sequence MLLRSDWYFDVW (SEQ ID NO:61). In some aspects, the heterologous insertion comprises the amino acid sequence LRYGRQYS (SEQ ID NO:10). In some aspects, the heterologous insertion comprises the amino acid sequence NVYAMTRNWWSDVY (SEQ ID NO: 11), NVWHDLVWSVSTTDVY (SEQ ID NO:12), NVYARPRPDNLNWSDVY (SEQ ID NO:13), HVETPYDSPELNWWDVY (SEQ ID NO:14), or NVYELVEDTSAYEIGVDVY (SEQ ID NO:15). In some aspects, the heterologous insertion comprises the amino acid sequence MLLRSDWYFDVW (SEQ ID NO:61), TGWGFDYW (SEQ ID NO:16), TRGATALDYW (SEQ ID NO:17), NVYAMTRNWWSDVY (SEQ ID NO:18), NVWHDLVWSVSTTDVY (SEQ ID NO: 19), NVYARPRPDNLNWSDVY (SEQ ID NO:20), HVETPYDSPELNWWDVY (SEQ ID NO:21), or NVYELVEDTSAYEIGVDVY (SEQ ID NO:22). In some aspects, the heterologous insertion comprises the amino acid sequence: ARGLSGNYV (SEQ ID NO: 23), ARGLSGNFV (SEQ ID NO:24), GLSGNYVMDY (SEQ ID NO:25), GLSGNYVVDY (SEQ ID NO:26), GLSGNFVMDF (SEQ ID NO:27) ARGTRA (SEQ ID NO: 28) GTRAYHY (SEQ ID NO:29), GTYRAYHF (SEQ ID NO:30) ARYGYGNPATRY (SEQ ID NO:31), YGYGNPATRYFDV (SEQ ID NO:32), TRGGYDSRAWF (SEQ ID NO:33), GGYDSRAWFAY (SEQ ID NO:34), GGYDSRAWFAH (SEQ ID NO:35), ARQGALYDGYYRGA (SEQ ID NO:36), QGALYDGYYRGAMDY (SEQ ID NO:37), ARRGYGYDGEF (SEQ ID NO:38), RGGYGYDGEFAY (SEQ ID NO:39), GLSGN (Y/F) V (M/V) D (Y/F) (SEQ ID NO:40), GTRAYH (Y/F) (SEQ ID NO:41) or (G/Q/R)G(Y/A/G)(D/L/Y)(S/Y/G)(R/D/Y)(A/G/D)(W/Y/G)(F/Y/E)(R/F/-)(G/-)(A/-)(M/-)(A/D)(Y/H) (SEQ ID NO:42). In some aspects, the heterologous insertion comprises the amino acid sequence: VRLRGYFDY (SEQ ID NO:43), ARRDGSYYPYYWYFDL (SEQ ID NO: 44), ARTLINYSDYADYVMDA (SEQ ID NO:45), ARSLINYRNYGDYVMDA (SEQ ID NO:46), ARNGVYHNYWYFDF (SEQ ID NO:47), or AKEGGWFLRIYGMDY (SEQ ID NO:48).

In some aspects of the antigen-binding proteins provided herein, the antigen-binding proteins further comprise a first linker at the N-terminus of the heterologous insertion and/or a second linker at the C-terminus of the heterologous insertion. In some aspects, the first linker comprises GGG and/or the second linker comprises GGG. In some aspects, the first linker comprises GGC and/or the second linker comprises CGG. In some aspects, the first linker comprises GGC and the second linker comprises CGG. In some aspects, the first linker and the second linker are capable of forming a cysteine bridge. In some aspects, the first is a proline-linker or a proline-rich linker and/or the second linker is a proline linker or a proline-rich linker.

In some aspects of the polypeptides, fusion proteins, and/or antigen-binding proteins provided herein, the polypeptide, fusion protein, or antigen-binding protein can be trancytosed across the blood brain barrier. In some aspects, the mechanism of transport across the blood brain barrier is receptor-mediated transport. In some aspects, at least 0.5% of the polypeptide, fusion protein, or antigen-binding protein that is injected intravenously into mice accumulates in vessel-depleted brain fraction of the mice. In some aspects, the polypeptide, fusion protein, or antigen-binding protein has a 3-100-fold increase in accumulation in vessel-depleted brain fraction of mice injected intravenously with the polypeptide, fusion protein, or antigen-binding protein as compared to a polypeptide, fusion protein, or antigen-binding protein that is identical except that it lacks the heterologous insertion.

In some aspects of antigen-binding proteins provided herein, the antigen-binding protein is an antibody. In some aspects, the antigen-binging proteins is an antigen-binding fragment of an antibody. In some aspects, the antigen-binding protein comprises an scFv. In some aspects, the antigen-binding protein is homodimeric. In some aspects, the antigen-binding protein is an IgG1 antigen-binding protein. In some aspects, the antigen-binding protein has a melting temperature (TM) similar to a wild-type IgG1 antibody. In some aspects, the antigen-binding protein has a TM of about 63° C. to about 75° C., optionally wherein the antigen-binding protein has a TM of about 67° C. to about 73° C. In some aspects, the antigen-binding protein has a TM that is similar to the TM of an antigen-binding protein that is identical except that it lacks the heterologous insertion. In some aspects, the antigen-binding protein binds to neonatal Fc receptor (FcRn). In some aspects, the antigen-binding protein binds to FcRn with a monomeric affinity of ≤1000 nM. In some aspects, the antigen-binding domain comprises an antigen-binding domain that specifically bind to a brain antigen. In some aspects, the brain antigen is beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), or caspase 6. In some aspects, the antigen-binding protein comprises the amino acid sequence of SEQ ID NO: 7.

In some aspects of the polypeptides, fusion proteins, and/or antigen-binding proteins provided herein, polypeptides, fusion proteins, and/or antigen-binding proteins further comprise a detectable label.

Provided herein are libraries comprising a plurality of polypeptides, fusion proteins, or antigen-binding proteins provided herein.

Provided herein are polynucleotides encoding a polypeptide, fusion protein, or antigen-binding protein provided herein.

Provided herein are expression system comprising a polynucleotide provided herein.

Provided herein are polynucleotides comprising a nucleic acid sequence encoding a polypeptide comprising an engineered constant domain, wherein the engineered constant domain comprises a heterologous insertion in a constant domain, wherein the heterologous insertion (i) comprises a CDRH3 or a target-binding fragment thereof and/or (ii) specifically binds to a BBB target. In some aspects, the polypeptide comprises an antibody heavy chain or fragment thereof. Provided herein are expression systems comprising a polynucleotide provided herein, optionally wherein the expression system further comprises a polynucleotide encoding an antibody light chain or fragment thereof.

Provided herein are pharmaceutical compositions comprising a polypeptide, fusion protein, antigen-binding protein, polynucleotide, or expression system provided herein and a pharmaceutically acceptable carrier.

Provided herein are libraries comprising a plurality of polynucleotides provided herein.

Provided herein are methods for making a polynucleotide comprising a nucleic acid sequence encoding an engineered constant domain comprising a heterologous insertion in a constant domain, the method comprising (a) providing a first nucleic acid sequence encoding a constant domain and (b) inserting a heterologous nucleic acid sequence into the region of the first nucleic acid sequence encoding the constant domain to produce the polynucleotide, wherein the heterologous nucleic acid sequence encodes (i) a CDRH3 or a target-binding fragment thereof and/or (ii) an amino acid sequence that binds to a BBB target.

Provided herein are methods for making a polynucleotide comprising a nucleic acid sequence encoding a fusion protein comprising an engineered constant domain comprising a heterologous insertion, the method comprising (a) providing a first nucleic acid sequence encoding a fusion protein comprising an engineered constant domain and (b) inserting a heterologous nucleic acid sequence into the region of the first nucleic acid sequence encoding the engineered constant domain to produce the polynucleotide, wherein the heterologous nucleic acid sequence encodes (i) a CDRH3 or a target-binding fragment thereof and/or (ii) an amino acid sequence that binds to a BBB target.

Provided herein are methods for making a polynucleotide comprising a nucleic acid sequence encoding an antigen-binding protein comprising an engineered constant domain comprising a heterologous insertion in a constant domain, the method comprising (a) providing a first nucleic acid sequence encoding an antigen-binding protein comprising a constant domain and (b) inserting a heterologous nucleic acid sequence into the region of the first nucleic acid sequence encoding the constant domain to generate the engineered constant domain of the polynucleotide, wherein the heterologous nucleic acid sequence encodes (i) a CDRH3 or a target-binding fragment thereof and/or (ii) an amino acid sequence that binds to a BBB target, thereby making the polynucleotide.

Provided herein are polynucleotides produced by a method provided herein. Provided herein are expression systems comprising a polynucleotide provided herein.

In some aspects of expression systems provided herein, the expression system is a host cell.

Provided herein are methods of producing an antigen-binding protein comprising culturing a host cell provided herein, optionally wherein the method further comprises recovering the antigen-binding protein from the culture.

Provided herein are methods for adding a binding specificity to a protein comprising a constant domain, the method comprising (a) providing a first nucleic acid sequence encoding a protein comprising a constant domain, (b) inserting a heterologous nucleic acid sequence into the first nucleic acid sequence to produce a modified polynucleotide, wherein the heterologous nucleic acid sequence encodes a CDRH3 or a target-binding fragment thereof, and (c) expressing the modified polynucleotide in an expression system.

Provided herein are methods method for increasing the ability of a protein comprising a constant domain to cross a blood brain barrier, the method comprising (a) providing a first nucleic acid sequence encoding a protein comprising the constant domain, (b) inserting a heterologous nucleic acid sequence into the region of the first nucleic acid sequence encoding the constant domain to produce a modified polynucleotide, wherein the heterologous nucleic acid sequence encodes an amino acid sequence that binds to a blood brain barrier target, and (c) expressing the modified nucleic acid sequence in an expression system. In some aspects, the amino acid sequence that binds to the blood brain barrier comprises a CDRH3 or a target-binding fragment thereof. In some aspects, the protein comprising the constant domain is an antigen-binding protein, optionally wherein the antigen-binding protein is an antibody or antigen-binding fragment thereof.

In some aspects of the methods provided herein, the CDRH3 was derived from an antibody or antigen-binding fragment thereof known to bind to a blood brain barrier target. In some aspects of the methods provided herein, the methods further comprise mutagenizing the heterologous nucleic acid sequence in the polynucleotide. In some aspects, the mutagenizing produces a library of polynucleotides comprising nucleic acid sequences encoding antigen-binding proteins comprising constant domains comprising heterologous insertions. In some aspects, a method provided herein further comprises screening the antigen-binding proteins encoded by the library for the ability to bind to a blood brain barrier protein and/or for the ability to cross the BBB.

Provided herein are methods for identifying a protein with the ability to cross a blood brain barrier, the method comprising (i) creating a library of nucleic acids encoding proteins, wherein the proteins comprise engineered constant domains that comprise heterologous insertions comprising CDRH3s or target-binding fragments thereof, (ii) expressing the proteins encoded by the library, and (iii) screening the proteins for the ability to bind to a BBB target and/or cross the BBB.

Provided herein are methods for producing a protein with the ability to cross a BBB, the method comprising (i) screening a library of proteins containing CDRH3 domains for the ability to bind a blood brain barrier target, (ii) selecting a protein from the library of proteins and identifying the CDRH3 domain of the selected protein, (iii) inserting a nucleic acid sequence encoding the CDRH3 domain or a target-binding fragment thereof of the selected protein into a nucleic acid sequence encoding a protein comprising a constant domain to produce a modified polynucleotide, wherein the insertion is within the region of the nucleic acid encoding the constant domain, and (iv) expressing the modified polynucleotide in a host cell. In some aspects, the library of proteins containing CDRH3 domains is a library comprising antibodies and/or antigen-binding fragments. In some aspects, the method further comprises determining the sequence of the CDRH3 domain.

Provided herein are methods for producing a protein with the ability to cross a BBB, the method comprising (i) inserting a nucleic acid sequence encoding a CDRH3 or a target-binding fragment thereof into a nucleic acid sequence encoding a protein comprising a constant domain to produce a polynucleotide, wherein the insertion is within the region of the nucleic acid encoding the constant domain, (ii) expressing the polynucleotide to produce a protein, and (iii) screening the protein for the ability to bind a blood brain barrier target and/or cross the blood brain barrier, optionally wherein the CDRH3 or target-binding fragment thereof is derived from an antibody.

In some aspects of the methods provided herein, the antigen-binding protein is an antibody or antigen-binding fragment thereof. In some aspects, the protein has a 3 to 100-fold increase in accumulation in vessel-depleted brain fraction of mice injected intravenously with the protein as compared to a protein that is identical except that it lacks the CDRH3 domain or target-binding fragment thereof.

Provided herein are methods for engineering a protein capable of crossing the BBB, the method comprising (i) providing a first nucleic acid encoding a protein, wherein the protein comprises a constant domain, (ii) inserting a heterologous nucleic acid encoding a CDRH3 domain or target-binding fragment thereof into the portion of the first nucleic acid that encodes the constant domain to produce a modified nucleic acid encoding a modified protein, (iii) expressing the modified nucleic acid in an expression system to produce the modified protein, and (iv) screening the modified protein for binding to a BBB target and/or for crossing the BBB. In some aspects, the protein is an antigen-binding protein, optionally wherein the antigen-binding protein is an antibody or antigen-binding fragment thereof. In some aspects, the protein has a 3 to 100-fold increase in accumulation in vessel-depleted brain fraction of mice injected intravenously with the protein as compared to a protein that is identical except that it lacks the CDRH3 domain or a target-binding fragment thereof.

Provided herein are proteins made using a method provided herein.

Provided herein are libraries of proteins made using a method provided herein.

Provided herein are methods of detecting an antigen in the central nervous system of a subject, the method comprising administering an antigen-binding protein provided herein to the subject and detecting the binding of the antigen-binding protein to the antigen in the central nervous system, optionally wherein the antigen is in the brain.

Provided herein are methods of transporting a polypeptide, fusion protein, antigen-binding protein across a BBB in a subject, the method comprising administering to the subject a polypeptide, fusion protein, or antigen-binding protein provided herein.

Provided herein are methods of treating a neurological disease or disorder in a subject comprising administering a polypeptide, fusion protein, or antigen-binding protein provided herein to the subject. In some aspects, the neurological disease or disorder is selected from a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some aspects, the neurological disease or disorder is selected from Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, traumatic brain injury, and limbic-predominant age-related TDP-43 encephalopathy (LATE). In some aspects, the dementia is frontotemporal dementia (FTD).

### BRIEF DESCRIPTION OF THE DRAWINGS/FIGURES

FIG. 1 shows binding of a molecule comprising an engineered constant domain comprising a heterologous CDRH3 sequence binds to a BBB target (e.g., a BBB receptor protein), and transport of the molecule across the BBB through receptor-mediated transcytosis.

FIG. 2 shows an exemplary insertion of a heterologous sequence (e.g., CDRH3 sequence) of a BBB-binding protein (antibody) into a CH3 domain of a second molecule (e.g., an antigen-binding protein or a fusion protein).

FIG. 3 shows exemplary locations of insertion of heterologous sequences (e.g., CDRH3 sequences) into antibodies.

FIG. 4 shows an exemplary method for generating a molecule comprising an engineered constant domain, wherein the engineered constant domain comprises a heterologous CDRH3 inserted in a constant domain. The method uses an antigen-binding protein that binds to a BBB target.

FIG. 5 shows another exemplary method for generating a molecule comprising an engineered constant domain, wherein the engineered constant domain comprises a heterologous CDRH3 inserted in a constant domain. The method uses a library of proteins that bind to one or more BBB targets.

FIG. 6 shows an exemplary method for generating a polynucleotide encoding an engineered constant domain, wherein the engineered constant domain comprises a heterologous CDRH3 inserted in a constant domain.

FIG. 7 shows the sequence of BLP1.1. (See Example 3.)

FIG. 8 shows binding of CDRH3-grafted antibodies to a target (human transferrin receptor (huTfR) protein). (See Example 2.)

FIG. 9 shows internalization of CDRH3-grafted antibodies over time. (See Example 4.)

FIG. 10 shows the results of assays measuring the binding of BLP1.1 to murine and cynomolgus transferrin receptor (TfR), to human FcRn, to huCD98hc, and to the apical domain of TfR. The assay is depicted on the right. (See Example 5.)

FIG. 11 shows that BLP1.1 does not change TfR levels in hCMEC/D3 cells. (See Example 6.)

FIG. 12 shows the melting temperature of BLP1.1 as determined by Differential Scanning Fluorimetry (DSF). TM1 was measured at 71.6° C., and TM2 was measured at 87.3° C. (See Example 7.)

### DETAILED DESCRIPTION OF THE INVENTION

### I. Definitions

The “central nervous system” or “CNS” refers to the complex of nerve tissues that control bodily function, and includes the brain and spinal cord.

The term “blood brain barrier” or “BBB” refers to a network of brain capillary endothelial cells that are closely sealed by tight junctions.

The term “blood brain barrier target” or “BBB target” refers to a molecule present on the endothelial cells of the BBB. A BBB target can be, for example, a protein (including a glycoprotein), sugar, or lipid. In some aspects, a BBB target is enriched on the endothelial cells of the BBB as compared to other cells of the body. In some aspects, a BBB target can be, e.g., a BBB receptor.

A “blood brain barrier receptor” or “BBB receptor” is a transmembrane receptor protein expressed on endothelial cells of the BBB, which is capable of transporting molecules across the blood-brain barrier. Examples of BBB receptors include, but are not limited to, transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), glucose transporter 1 (Glut1) and heparin-binding epidermal growth factor-like growth factor (HB-EGF). An exemplary BBB receptor herein is transferrin receptor (TfR).

A “central nervous system antigen” or “CNS antigen” is an antigen expressed in the CNS, including the brain, which can be targeted with an antibody or small molecule. Examples of such antigens include, without limitation: beta-secretase 1 (BACE1), amyloid beta (Abeta), epidermal growth factor receptor (EGFR), human epidermal growth factor receptor 2 (HER2), tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), TNF receptor 1 (TNFR1), interleukin 1 beta (IL1 (3)), and caspase 6. In one aspect, the antigen is BACE1. A “brain antigen” is an antigen expressed in the brain.

As used herein, the terms “antibody” and “immunoglobulin” are used interchangeably and refer to an antibody molecule that recognizes and specifically binds to a target, such as a protein, polypeptide, peptide, carbohydrate, polynucleotide, lipid, or combinations of the foregoing (e.g., a glycoprotein), through at least one antigen recognition site within the variable region of the immunoglobulin molecule. The term “antibody” encompasses monoclonal antibodies, chimeric antibodies, humanized antibodies, human antibodies, and any other immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1, and IgA2), based on the identity of their heavy-chain constant domains referred to as alpha, delta, epsilon, gamma, and mu, respectively. The different classes of antibodies have different and well known subunit structures and three-dimensional configurations. Antibodies can be naked, part of a fusion protein, or conjugated to other molecules such as toxins, radioisotopes, etc.

The term “antibody fragment” refers to a portion of an antibody. An “antigen-binding fragment,” “antigen-binding domain,” or “antigen-binding region,” refers to a portion of an antibody that binds to an antigen. An antigen-binding fragment can contain the antigenic determining regions of an antibody (e.g., the complementarity determining regions (CDRs)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The term “antigen” or “target” as used herein refers to molecules or structures known to interact or capable of interacting with one or more antibody CDR-loops, including an antibody CDRH3 domain or fragment thereof.

The terms “monoclonal” antibody or antigen-binding fragment” thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in a sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs). The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDRH1), amino acid positions 50 to 65 (CDRH2), and amino acid positions 95 to 102 (CDRH3). Using the Kabat numbering system, CDRs within an antibody light chain molecule are typically present at amino acid positions 24 to 34 (CDRL1), amino acid positions 50 to 56 (CDRL2), and amino acid positions 89 to 97 (CDRL3). In some aspects, the CDRs of the antibodies described herein have been determined according to the Kabat numbering scheme. Chothia refers instead to the location of the structural loops (Chothia and Lesk, J. Mol. Biol. 196:901-917 (1987)). The end of the Chothia CDRH1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The terms “VH” and “VH domain” are used interchangeably to refer to the heavy chain variable region of an antibody.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄. Heavy chain amino acid sequences are well known in the art. In some aspects, the heavy chain is a human heavy chain.

The terms “VL” and “VL domain” are used interchangeably to refer to the light chain variable region of an antibody.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

As used herein, the term “constant region” is a region of an antibody that is not the variable region of the antibody, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

An “engineered constant region” refers to a non-naturally occurring constant region that has been designed or arranged, e.g., to comprise a heterologous sequence that binds to a BBB target and/or to comprise a heterologous CDRH3.

A “constant domain” means a domain within a constant region that is capable of forming an immunoglobulin fold. Constant domains include the CH1, CH2, CH3, and CL domains.

An “engineered constant domain” refers to a non-naturally occurring constant domain that has been designed or arranged, e.g., to comprise a heterologous sequence that binds to a BBB target and/or to comprise a heterologous CDRH3. For example, an engineered constant domain comprising a CH3 domain can refer to a CH3 domain that comprises a heterologous sequence that binds to a BBB target when present within the CH3 domain.

Antibodies are made of domains that form immunoglobulin folds. Anti-parallel beta sheets are connected by loops to form a compressed antiparallel beta barrel. In the variable region, some of the loops of the domains contribute essentially to the specificity of the antibody, i.e. the binding to an antigen. These loops are called CDR-loops. All other loops are defined herein as “structural loops.”

The terms “fragment crystallizable region,” “Fc region,” “Fc” and refer to an antibody region comprising the hinge, CH2, and CH3 domains. These terms include molecules in monomer and multimer form. These terms encompass molecules digested from a whole antibody or produced by other means.

The term “multimer” as applied to constant domains or molecules comprising constant domains refers to molecules comprising two or more polypeptide chains associated covalently, non-covalently, or by both covalent and non-covalent interactions. IgG and IgD molecules can form dimers. IgM molecules can form pentamers. IgA molecules can form monomers, dimers, trimers, or tetramers. Multimers can be formed by exploiting the sequence and resulting activity of the native Ig source of the Fc or by the native Fc. The term “dimer” as applied to constant domains or molecules comprising constant domains refers to molecules having two polypeptide chains associated covalently or non-covalently.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g. murine) sequences. Typically, humanized antibodies or antigen-binding fragments thereof are human immunoglobulins in which residues from the complementarity determining regions (CDRs) are replaced by residues from the CDRs of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). The humanized antibody or antigen-binding fragment thereof can be further modified by the substitution of additional residues either in the Fv framework region and/or within the replaced non-human residues to refine and optimize the specificity, affinity, and/or capability of the antibody or antigen-binding fragment thereof. In general, the humanized antibody or antigen-binding fragment thereof will comprise VH and VL that comprise substantially all of at least one, and typically two or three, of the CDR regions that correspond to the non-human immunoglobulin, whereas all or substantially all of the FR regions are those of a human immunoglobulin consensus sequence. The humanized antibody or antigen-binding fragment thereof can also comprise at least a portion of an immunoglobulin constant region or Fc region, typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some aspects, a “humanized antibody” is a resurfaced antibody. The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation rate constant of, e.g., an antibody or antigen-binding fragment thereof from an antigen. The kₒₙ and k_{off} can be determined by techniques known to one of ordinary skill in the art, such as BIAcore® or KinExA.

An antibody that “downregulates” the antigen to which it binds refers to an antibody that reduces expression of the antigen on a cell surface.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, comprise non-contiguous amino acids of a polypeptide or two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., alanine scanning or other site-directed mutagenesis mapping).

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails complementarity between the antigen binding domain and the epitope.

An antibody that “binds to the same epitope” as a reference antibody refers to an antibody that contacts the same amino acid residues on the antigen as the reference antibody. The ability of an antibody to bind to the same epitope as a reference antibody can be determined using peptide scanning mutagenesis or high throughput alanine scanning mutagenesis. In the latter methodology, a comprehensive mutation library of antigen, or a portion thereof (e.g., the extracellular domain), can be generated by mutating each individual amino acid residue to alanine (or if the amino acid residue is alanine, then to another residue such as serine) and testing each mutant for binding to a target antibody or antigen binding fragment thereof.

An antibody is said to “competitively inhibit” binding of a reference antibody to a given epitope if it preferentially binds to that epitope or an overlapping epitope such that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

A molecule that “blocks” a function refers to a molecule that prevents any detectable function from occurring. Thus, for example, a protein that “blocks” iron transport prevents iron transport from occurring. A protein that does not block iron transport can decrease or reduce iron transport, but does not prevent it.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

The term “linker” or “linked” refers to the covalent linkage between two polypeptides or two heterologous molecules. In certain aspects, a linker is a chemical linker. In certain aspects, the linker comprises a peptide bond and the two polypeptides or two heterologous molecules are linked to each other either directly to or via one or more additional amino acids. The composition and length of the linker can be determined in accordance with methods well known in the art and can be tested for efficacy. The linker can be from about 3 to about 15 amino acids long, in some aspects about 5 to about 10 amino acids long, however, longer or shorter linkers may be used or the linker may be dispensed with entirely. A glycine linker is one that comprises one or more glycines but no other amino acids, e.g., GGGG. A glycine-rich linker is one that comprises one or more glycines and can contain other amino acids as long as glycine is the predominant species in the linker e.g., GGGNGG. A glycine-serine linker is one which contains both glycine and serine in any proportion, e.g., GGGS. Similarly, a proline linker is one that comprises one or more prolines but no other amino acids. A proline-rich linker is one that comprises one or more prolines and can contain other amino acids so long as proline is the predominant species in the linker.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the Blast program set at default parameters, and alignment of amino acid sequences can be performed with the Blast program set at default parameters (see National Center for Biotechnology Information (NCBI) on the worldwide web, ncbi.nlm.nih.gov).

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “expression system” refers to one or more nucleic acid molecules comprising coding sequence and control sequence(s) in operable linkage, along with a host cell and/or other in vitro transcription and translation machinery, such that one or more proteins encoded by the nucleic acid molecule(s) are capable of being produced.

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that may be used to deliver a drug, e.g., an anti-human antibody or antigen-binding fragment thereof, to the desired site of biological action.

As used herein, the terms “subject” and “patient” are used interchangeably. The subject can be a mammal such as a non-human animal (e.g., cow, pig, horse, cat, dog, rat, mouse, monkey or other primate, etc.). In some aspects, the subject is a cynomolgus monkey. In some aspects, the subject is a human.

The term “therapeutically effective amount” refers to an amount of a drug, e.g., an anti-human antibody or antigen-binding fragment thereof, effective to treat a disease or condition in a subject.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

### II. Constant Domains Comprising Heterologous Insertions

### Exemplary Methods

Methods are provided herein for generating a molecule capable of binding to a specific BBB target and transporting the molecule across the BBB.

Provided herein are methods for generating an engineered constant domain (or a polypeptide or antigen-binding protein comprising an engineered constant domain), wherein the engineered constant domain comprises a heterologous insertion in a constant domain, and wherein the heterologous insertion is capable of binding a BBB target. In one aspect, the heterologous insertion is binding domain that binds to a BBB target. In a particular aspect, the heterologous insertion is a CDRH3 sequence that is specific for the BBB target.

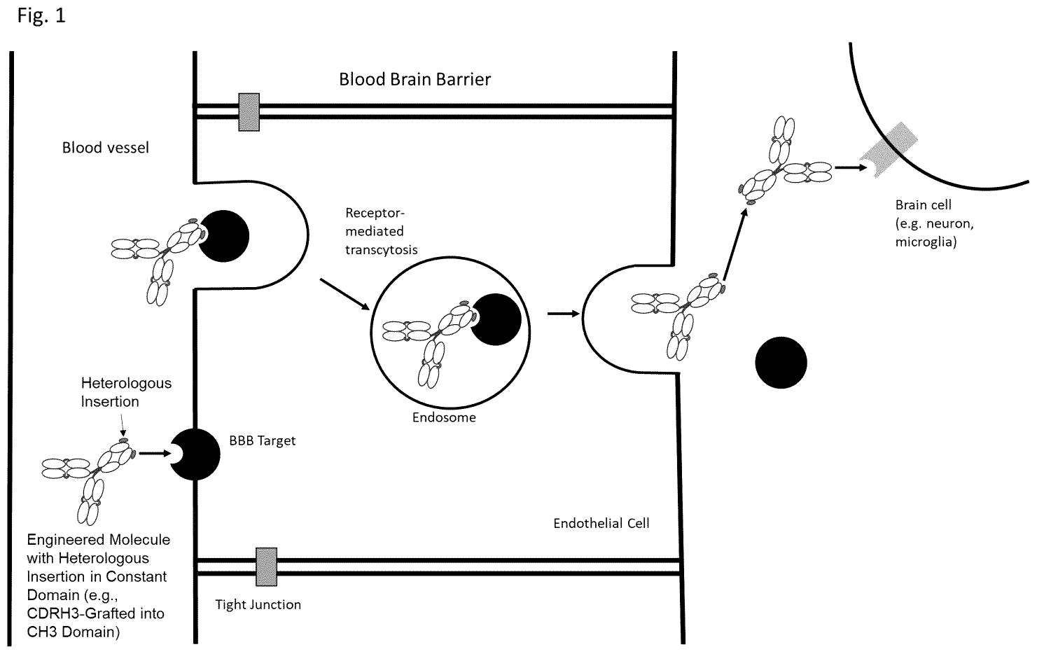

Further provided herein are methods for transporting engineered molecules comprising one or more engineered constant domains across the BBB. For example, as shown in FIG. 1, an engineered molecule with an engineered constant domain comprising a heterologous sequence binds to a BBB target (e.g., a BBB receptor protein), and the engineered molecule comprising the engineered constant domain is transported across the BBB through receptor-mediated transcytosis. In some aspects, the heterologous sequence is a CDRH3 sequence that binds to a BBB target.

In some aspects, the method comprises identifying one or more antigen-binding proteins that bind to a selected BBB target (“BBB-binding protein”) and determining the sequences of the BBB-binding domain of the BBB-binding proteins. In one aspect, the BBB-binding domain comprises a CDRH3 sequence. In one example, a CDRH3 sequence (BBB-binding domain) of an anti-transferrin receptor antibody (BBB-binding protein) that specifically binds transferrin receptor (BBB target) is identified.

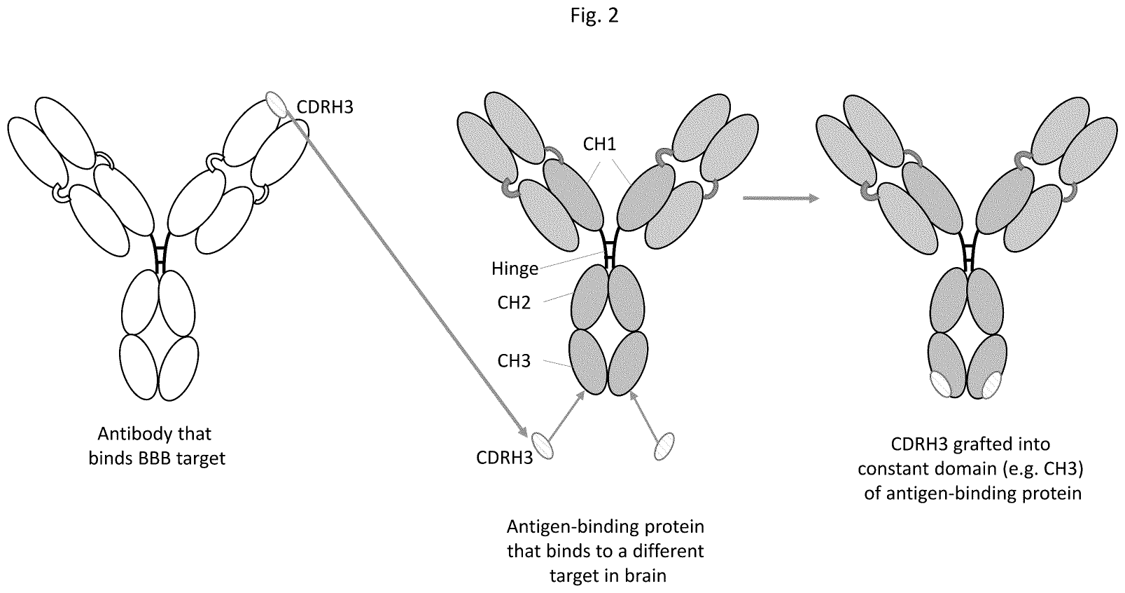

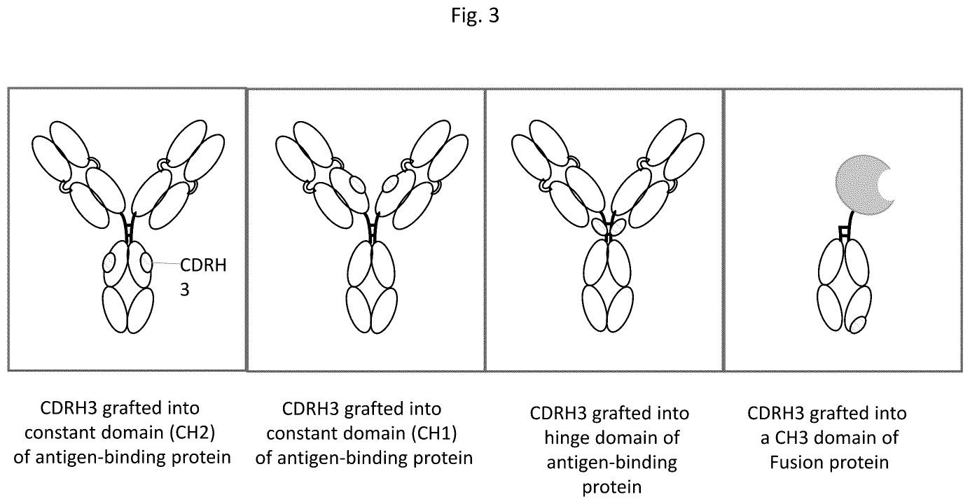

Methods are provided for engineering a molecule (e.g. a polypeptide, a fusion protein or an antigen-binding protein) comprising an engineered constant domain comprising a heterologous insertion. FIG. 2 shows an example method for inserting a heterologous sequence (e.g., CDRH3 sequence) of a BBB-binding protein into a CH3 domain of a second molecule (e.g., an antigen-binding protein or a fusion protein). In some aspects, the engineered molecule is also capable of binding to a different target in the brain (a target other than the BBB target). Heterologous insertions (e.g., CDRH3 sequences) can be inserted into other locations as well, e.g., as shown in FIG. 3. It is understood that in any of the embodiments in FIG. 2 and FIG. 3, which show dimeric structures, insertion of the heterologous sequence may occur in only one of two corresponding constant domains to form an asymmetric molecule, as exemplified in FIG. 3, far right panel.

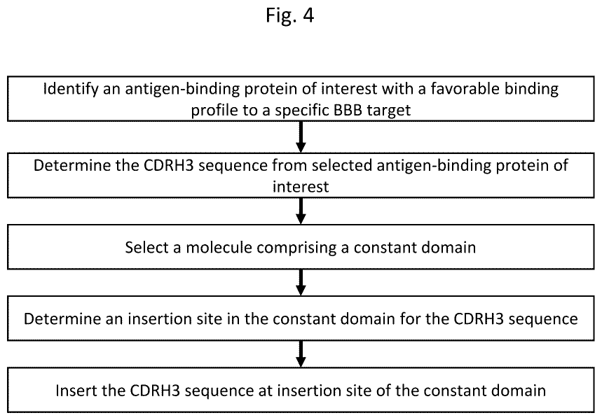

As shown in FIG. 4, also provided herein are methods comprising identifying an antigen-binding protein of interest with a favorable binding profile to a specific BBB target, determining the CDRH3 sequence from a selected antigen-binding protein of interest, selecting a molecule comprising a constant domain, determining an insertion site in the constant domain for insertion of the CDRH3 sequence, and inserting the CDRH3 sequence at the insertion site of the constant domain. In some aspects, the method further comprises determining the binding characteristics of the engineered constant domain to a BBB target and/or determining the ability of the engineered constant domain to cross the BBB.

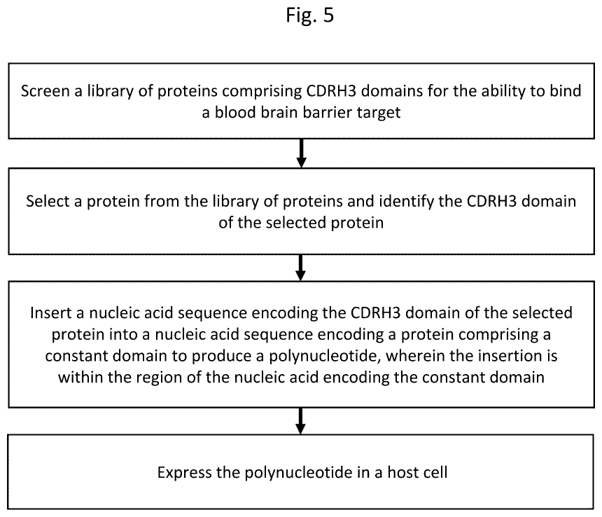

Provided herein are methods for screening a library of proteins comprising CDRH3 domains for the ability to bind a blood brain barrier target, selecting a protein from the library of proteins and identifying the CDRH3 domain of the selected protein, inserting a nucleic acid sequence encoding the CDRH3 domain of the selected protein into a nucleic acid sequence encoding a protein comprising a constant domain to produce a polynucleotide, wherein the insertion is within the region of the nucleic acid encoding the constant domain, and expressing the polynucleotide in a host cell. See FIG. 5.

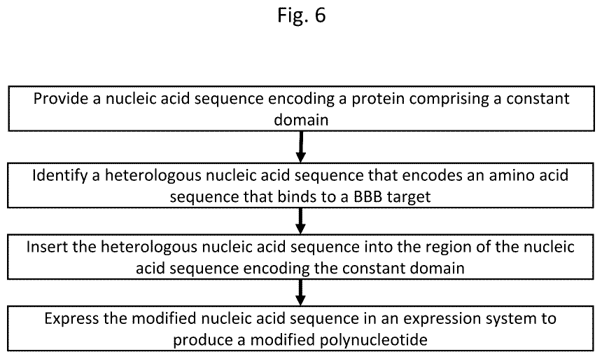

Provided herein are methods for providing a nucleic acid sequence encoding a protein comprising a constant domain, identifying a heterologous nucleic acid sequence that encodes an amino acid sequence that binds to a BBB target, inserting the heterologous nucleic acid sequence into the region of the nucleic acid sequence encoding the constant domain, and expressing the modified nucleic acid sequence in an expression system to produce a modified polynucleotide. See FIG. 6.

The methods provided herein can also be used to insert heterologous sequences to confer binding specificity to any target of interest.

### Exemplary Compositions

Compositions are provided herein for molecules comprising engineered constant domains that are capable of binding to a BBB target and crossing the BBB. In some aspects, an engineered constant domain (or a polypeptide or an antigen-binding protein comprising an engineered constant domain) is provided, wherein the engineered constant domain comprises a heterologous insertion in a constant domain, and wherein the heterologous insertion is capable of binding a BBB target. In some aspects, the heterologous insertion is a binding domain that binds to a BBB target. In some aspects, the heterologous insertion is a CDRH3 sequence that is specific for the BBB target.

Domains in antibody molecules have a similar structure of two beta sheets packed tightly against each other in a compressed antiparallel beta barrel. This conserved structure is termed the immunoglobulin fold. The immunoglobulin fold of constant and variable domains generally contains a 3-stranded sheet packed against a 4-stranded sheet. The fold is stabilized by hydrogen bonding between the beta strands of each sheet, by hydrophobic bonding between residues of opposite sheets in the interior, and by a disulfide bond between the sheets. The 3-stranded sheet comprises strands C, F, and G, and the 4-stranded sheet has strands A, B, E, and D. The letters A through G denote the sequential positions of the beta strands along the amino acid sequence of the immunoglobulin fold. Amino acids connecting the beta strands form loops. The variable domains of both light and heavy immunoglobulin chains contain three hypervariable loops, or complementarity-determining regions (CDRs). The loops which are not CDR-loops, e.g., loops in the constant domains of native immunoglobulins, are called structural loops.

In some aspects, a polypeptide comprising a constant domain comprises one or more structural loops. Exemplary structural loops are provided in WO 2006/072620, which is herein incorporated by reference in its entirety. In some aspects, an AB loop of a CH3 corresponds to amino acids 242-244 of a native (wild type or “WT”) hIgG1 constant region sequence (SEQ ID NO:3; amino acids 359-361 according to EU numbering). In some aspects, a CD loop of a CH3 corresponds to amino acids 269-272 of SEQ ID NO:3 (amino acids 386-389 according to EU numbering). In some aspects, an EF loop of a CH3 corresponds to amino acids 296-298 and 301-302 of SEQ ID NO:3 (amino acids 413-415 and 418-419 according to EU numbering). In some aspects, a heterologous sequence is inserted within a structural loop, at the N-terminus of the structural loop, or at the C-terminus of the structural loop.

In some aspects, a constant domain comprises a CH3 domain. In some aspects, a constant domain comprises a CH2 domain. In some aspects, a constant domain comprises a CH1 domain. In some aspects, a constant domain comprises a CL domain.

In some aspects, a constant domain thereof comprises a heterologous insertion in a structural loop of a variable domain. The structural loop of a human variable domain can comprise, for example, amino acids 8 to 20, amino acids 44 to 50, amino acids 67 to 76, and/or amino acids 89 to 101, wherein the numbering of the amino acid sequences of the immunoglobulin is according to the IMGT numbering scheme.

Various types of molecules can be engineered by inserting or grafting a heterologous CDRH3 sequence into a constant domain, as described herein. In some aspects, a polypeptide comprising an engineered constant domain comprises a CH2 domain and a CH3 domain. In some aspects, a polypeptide comprising an engineered constant domain comprises an Fc domain or fragment thereof. In some aspects, a polypeptide comprising an engineered constant domain comprises a CH1 domain, a hinge domain, a CH2 domain, and a CH3 domain. FIG. 2 shows an example of a modified constant domain comprising a heterologous insertion (e.g., CDRH3 sequence) in its CH3 domain. In other aspects, the modified constant domain comprises a heterologous insertion (e.g., CDRH3 sequence) in its CH2 domain. See FIG. 3. In other aspects, the modified constant domain comprises a heterologous insertion (e.g., CDRH3 sequence) in its CH1 domain. In other aspects, the modified constant domain comprises a heterologous insertion (e.g., CDRH3 sequence) in its CL domain. In another aspect, a polypeptide (e.g., an antibody or antigen-binding fragment thereof) comprises a heterologous insertion (e.g., CDRH3 sequence) in the hinge domain. In yet another aspect, a fusion molecule is engineered to have a modified Fc domain comprising a heterologous CDRH3 sequence and a heterologous polypeptide.

In some aspects, a constant domain is a human constant domain. In some aspects, an constant domain is a murine, rat, rabbit, or monkey (e.g., cynomolgus) constant domain.

In some aspects, a constant domain is an IgG constant domain. In some aspects, the IgG is an IgG1, IgG2, or IgG4. In some aspects, the IgG is an IgG1. In some aspects, the IgG is an IgG2. In some aspects, the IgG is an IgG4. In some aspects, an Fc domain or fragment thereof is an IgG Fc domain or fragment thereof.

In some aspects, a protein comprising a constant domain or Fc domain or fragment thereof comprises the amino acid sequence of SEQ ID NO:3 or a fragment thereof. In some aspects, a protein comprising a constant domain or Fc domain or fragment thereof comprises the CH2 domain of SEQ ID NO:3 or a fragment thereof. In some aspects, a protein comprising a constant domain or fragment thereof comprises the CH3 domain of SEQ ID NO:3 or a fragment thereof. In some aspects, a protein comprising a constant domain comprises the CH2 domain of SEQ ID NO:3 or a fragment thereof and the CH3 domain of SEQ ID NO:3 or a fragment thereof.

In some aspects, a protein comprising a constant domain comprises an amino acid sequence at least 70%, at least 75%, at least 80%, at least 85%, at least 90%, at least 95%, at least 96%, at least 97%, at least 98%, or at least 99% identical to SEQ ID NO:3. In some aspects, a protein comprising a constant domain comprises an amino acid sequence at least 70%, at least 75%, at least 80%, at least 85%, at least 90%, at least 95%, at least 96%, at least 97%, at least 98%, or at least 99% identical to CH2 domain of SEQ ID NO:3. In some aspects, a protein comprising a constant domain comprises an amino acid sequence at least 70%, at least 75%, at least 80%, at least 85%, at least 90%, at least 95%, at least 96%, at least 97%, at least 98%, or at least 99% identical to the CH3 domain of SEQ ID NO:3. In some aspects, a protein comprising a constant domain comprises an amino acid sequence at least 70%, at least 75%, at least 80%, at least 85%, at least 90%, at least 95%, at least 96%, at least 97%, at least 98%, or at least 99% identical to the CH2 domain of SEQ ID NO: 3 and an amino acid sequence at least 70%, at least 75%, at least 80%, at least 85%, at least 90%, at least 95%, at least 96%, at least 97%, at least 98%, or at least 99% identical to the CH3 domain of SEQ ID NO:3.

As provided herein, a constant domain can be engineered to comprise a heterologous insertion. Such a heterologous insertion can be within a structural loop, at the N-terminus of the structural loop, or at the C-terminus of the structural loop. Such a heterologous insertion can be within a CH2 domain Such a heterologous insertion can be within a CH3 domain. In some aspects, a heterologous insertion is in a structural loop region of a CH3 domain, wherein the structural loop regions of the CH3 comprise amino acids 7 to 21, amino acids 25 to 39, amino acids 41 to 81, amino acids 83 to 85, amino acids 89 to 103, and amino acids 106 to 117, wherein the numbering of the amino acid sequences is according to the IMGT numbering scheme. Such a heterologous insertion can be between a CH2 domain and a CH3 domain. Such a heterologous insertion can be within a CH1 domain, CL domain or hinge.

In some aspects, a heterologous insertion provided herein is between N384 and G385 of a CH3 domain or fragment thereof according to EU numbering of the full length hIgG1 sequence (i.e., amino acids 267 and 268 of SEQ ID NO:3). In some aspects, a heterologous insertion provided herein replaces N384 of a constant domain or fragment thereof according to EU numbering (i.e., amino acid 267 of SEQ ID NO:3). In some aspects, a heterologous insertion provided herein replaces G385 of a constant domain according to EU numbering (i.e., amino acid 268 of SEQ ID NO:3). In some aspects, a heterologous insertion provided herein replaces N384 and G385 of a constant domain according to EU numbering (i.e., amino acids 267 and 268 of SEQ ID NO:3).

In some aspects, a heterologous insertion provided herein is between L358 and T359 of a CH3 domain according to EU numbering of the full length hIgG1 sequence (i.e., amino acids 241 and 242 of SEQ ID NO:3). In some aspects, a heterologous insertion provided herein replaces L358 of a constant domain according to EU numbering (i.e., amino acid 241 of SEQ ID NO:3). In some aspects, a heterologous insertion provided herein replaces T359 of a constant domain according to EU numbering (i.e., amino acid 242 of SEQ ID NO:3). In some aspects, a heterologous insertion provided herein replaces L358 and T359 of a constant domain according to EU numbering (i.e., amino acids 241 and 242 of SEQ ID NO:3).

In some aspects, a heterologous insertion provided herein is in a loop connecting beta strands A-B and E-F of a human IgG1 CH3 domain.

In some aspects, a heterologous insertion is within a CH1 domain.

In some aspects, a heterologous insertion is within a CL domain.

In some aspects, a heterologous insertion is at the N-terminus of a CH1 domain, a CH2 domain, a CH3 domain, or a CL domain. In some aspects, a heterologous insertion is at the C-terminus of a CH1 domain, a CH2 domain, a CH3 domain, or a CL domain.

In some aspects, a polypeptide comprising a constant domain comprises at least 10 amino acids, at least 20 amino acids, at least 25 amino acids, at least 50 amino acids, at least 60 amino acids, at least 70 amino acids, at least 80 amino acids, at least 90 amino acids, at least 100 amino acids, or at least 150 amino acids. In some aspects, a polypeptide comprising a constant domain comprises about 10 to about 400 amino acids, about 20 to about 400 amino acids, about 25 to about 400 amino acids, about 50 to about 400 amino acids, about 60 to about 400 amino acids, about 70 to about 400 amino acids, about 80 to about 400 amino acids, about 90 to about 400 amino acids, about 100 to about 400 amino acids, or about 150 to about 400 amino acids.

Also provided herein are libraries comprising constant domains comprising heterologous insertions as described herein and libraries comprising polypeptide comprising such engineered constant domains.

### III. Heterologous Insertions

In some aspects, the heterologous insertion is a BBB-binding domain. In some aspects, the heterologous insertion is a CDRH3 or a target-binding fragment thereof. In some aspects, the heterologous insertion is a CDRH3 or a target-binding fragment thereof, wherein the CDRH3 or fragment thereof is capable of binding to a BBB target.

In certain aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDRH1 loop is present at heavy chain amino acids 26 to 32, 33, or 34, the Chothia CDRH2 loop is present at heavy chain amino acids 52 to 56, and the Chothia CDRH3 loop is present at heavy chain amino acids 95 to 102, while the Chothia CDRL1 loop is present at light chain amino acids 24 to 34, the Chothia CDRL2 loop is present at light chain amino acids 50 to 56, and the Chothia CDRL3 loop is present at light chain amino acids 89 to 97. The end of the Chothia CDRH1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, the CDRs of an antibody or antigen-binding fragment thereof can be determined according to the IMGT numbering system as described in Lefranc M-P, (1999) The Immunologist 7:132-136 and Lefranc M-P et al., (1999) Nucleic Acids Res 27:209-212. According to the IMGT numbering scheme, CDRH1 is at positions 26 to 35, CDRH2 is at positions 51 to 57, CDRH3 is at positions 93 to 102, CDRL1 is at positions 27 to 32, CDRL2 is at positions 50 to 52, and CDRL3 is at positions 89 to 97.

In certain aspects, the CDRs of an antibody or antigen-binding fragment thereof can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an antibody or antigen-binding fragment thereof can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.).

The CDRH3 domain can be a Kabat-defined, a Chothia-defined, an IMGT-defined, or an AbM-defined CDRH3. In some aspects, the CDRH3 is a Kabat-defined CDRH3. In some aspects, the CDRH3 is an AbM-defined CDRH3.

As described above, a CDRH3 can be identified from an antibody that binds to a target of interest. The CDRH3 can be grafted into a constant domain as described herein to confer new binding specificity, such that the constant domain is capable of binding the target of interest to which it would not otherwise bind. The CDRH3 can be further mutagenized after grafting to improve binding to the target of interest. In some aspects, the target of interest is a BBB target.

In some aspects, a BBB target is a protein, sugar, lipid, or glycoprotein present at the endothelial cells comprising the blood-brain barrier. In some aspects, it is enriched at the BBB and/or is capable of transporting across the BBB. In some aspects, a BBB target is a receptor protein. In some aspects, a BBB target is a lipid. In some aspects, a BBB target is a sugar. In some aspects, a BBB target is a glycoprotein.

In some aspects, a heterologous insertion (e.g., a CDRH3 or a target-binding fragment thereof, or an amino acid sequence that binds to a BBB target (a “BBB-binding domain”)) binds to a blood brain barrier target with an affinity of about 1 nM to about 1000 nM.

In some aspects, a heterologous insertion is capable of binding to transferrin receptor (e.g., Uniprot P02786), Basigin (e.g., Uniprot P35613), Glucose Transporter Type 1 (Glut1; e.g., Uniprot P11166), or CD98 (e.g., Uniprot P08195), MfsD2a (e.g., Uniprot Q8NA29), LRP1 (e.g., Uniprot Q07954), LRP8 (e.g., Uniprot Q14114), or IGF1R (e.g., Uniprot P08069). In some aspects, a heterologous insertion is capable of binding to the extracellular domain of transferrin receptor (e.g., Uniprot P02786), Basigin (e.g., Uniprot P35613), Glucose Transporter Type 1 (Glut1; e.g., Uniprot P11166), or CD98 (e.g., Uniprot P08195), MfsD2a (e.g., Uniprot Q8NA29), LRP1 (e.g., Uniprot Q07954), LRP8 (e.g., Uniprot Q14114), or IGF1R (e.g., Uniprot P08069).

In some aspects, a heterologous insertion is capable of binding to human transferrin receptor (TfR). The amino acid sequence of human transferrin receptor protein 1 is:

(SEQ ID NO: 9)
MMDQARSAFSNLFGGEPLSYTRFSLARQVDGDNSHVEMKLAVDEEE
NADNNTKANVTKPKRCSGSICYGTIAVIVFFLIGFMIGYLGYCKG
VEPKTECERLAGTESPVREEPGEDFPAARRLYWDDLKRKLSEKLD
STDFTGTIKLLNENSYVPREAGSQKDENLALYVENQFREFKLSKV
WRDQHFVKIQVKDSAQNSVIIVDKNGRLVYLVENPGGYVAYSKAA
TVTGKLVHANFGTKKDFEDLYTPVNGSIVIVRAGKITFAEKVANA
ESLNAIGVLIYMDQTKFPIVNAELSFFGHAHLGTGDPYTPGFPSF
NHTQFPPSRSSGLPNIPVQTISRAAAEKLFGNMEGDCPSDWKTDS
TCRMVTSESKNVKLTVSNVLKEIKILNIFGVIKGFVEPDHYVVVG
AQRDAWGPGAAKSGVGTALLLKLAQMFSDMVLKDGFQPSRSIIFA
SWSAGDFGSVGATEWLEGYLSSLHLKAFTYINLDKAVLGTSNFKV
SASPLLYTLIEKTMQNVKHPVTGQFLYQDSNWASKVEKLTLDNAA
FPFLAYSGIPAVSFCFCEDTDYPYLGTTMDTYKELIERIPELNKV
ARAAAEVAGQFVIKLTHDVELNLDYERYNSQLLSFVRDLNQYRAD
IKEMGLSLOWLYSARGDFFRATSRLTTDFGNAEKTDRFVMKKLND
RVMRVEYHFLSPYVSPKESPFRHVFWGSGSHTLPALLENLKLRKQ
NNGAFNETLFRNQLALATWTIQGAANALSGDVWDIDNEF

Amino acids 1-67 of SEQ ID NO:9 are the cytoplasmic domain of human transferrin receptor protein 1. Amino acids 68-88 of SEQ ID NO:9 are the transmembrane domain of human transferrin receptor protein 1, and amino acids 89-760 of SEQ ID NO:9 are the extracellular domain of human transferrin receptor protein 1. The extracellular domain comprises a protease-like domain, an apical domain, and a helical domain involved in homodimerization. The protease-like domain comprises amino acids residues 121-188 and 384-606) of SEQ ID NO:9. The apical domain comprises amino acids 189-383 of SEQ ID NO: 9. The helical domain comprises amino acids 607-760 of SEQ ID NO:9. In some aspects, a heterologous insertion is capable of binding an epitope within amino acids 68-88 of SEQ ID NO:9. In some aspects, a heterologous insertion is capable of binding to an epitope within amino acids 89-760 of SEQ ID NO:9. In some aspects, a heterologous insertion is capable of binding an epitope within amino acids 121-188 or 384-606 of SEQ ID NO: 9. In some aspects, a heterologous insertion is capable of binding an epitope within amino acids 189-383 of SEQ ID NO:9. In some aspects, a heterologous insertion is capable of binding an epitope within amino acids 196-379 of SEQ ID NO:9. In some aspects, a heterologous insertion is capable of binding an epitope within amino acids 607-760 of SEQ ID NO: 9.

In some aspects, a heterologous insertion (e.g., a CDRH3 or a target-binding fragment thereof or a BBB-binding domain) is capable of binding to murine TfR. In some aspects, a heterologous insertion is capable of binding to monkey (e.g., cynomolgus) TfR. In some aspects, a heterologous insertion is capable of binding to human and murine TfR. In some aspects, a heterologous insertion is capable of binding to human and monkey (e.g., cynomolgus) TfR. In some aspects, a heterologous insertion is capable of binding to human, murine, and monkey (e.g., cynomolgus) TfR.

In some aspects, a heterologous insertion (e.g., a CDRH3 or a target-binding fragment thereof or a BBB-binding domain) that binds to transferrin receptor does not block iron transport. In some aspects, a heterologous insertion that binds to transferrin receptor does not decrease iron transport by more than 50% (e.g., as compared to iron transport in the absence of the heterologous insertion). In some aspects, a heterologous insertion that binds to transferrin receptor does not decrease iron transport by more than 25% (e.g., as compared to iron transport in the absence of the heterologous insertion). In some aspects, a heterologous insertion that binds to transferrin receptor does not decrease iron transport by more than 10% (e.g., as compared to iron transport in the absence of the heterologous insertion). In some aspects, a heterologous insertion that binds to transferrin receptor does not decrease iron transport by more than 5% (e.g., as compared to iron transport in the absence of the heterologous insertion).

In some aspects, a heterologous insertion (e.g., a CDRH3 or a target-binding fragment thereof or a BBB-binding domain) that binds to transferrin receptor does not reduce the level of TfR-expressing peripheral cells by more than 15% (e.g., as compared to iron transport in the absence of the heterologous insertion). In some aspects, a heterologous insertion that binds to transferrin receptor does not reduce the level of TfR-expressing peripheral cells by more than 10% (e.g., as compared to iron transport in the absence of the heterologous insertion). In some aspects, a heterologous insertion that binds to transferrin receptor does not reduce the level of TfR-expressing peripheral cells by more than 5% (e.g., as compared to iron transport in the absence of the heterologous insertion).

In some aspects, a heterologous insertion (e.g., a CDRH3 or a target-binding fragment thereof or a BBB-binding domain) that binds to transferrin receptor comprises the amino acid sequence MLLRSDWYFDVW (SEQ ID NO:61).

In some aspects, a heterologous insertion (e.g., a CDRH3 or a target-binding fragment thereof or a BBB-binding domain) that binds to transferrin receptor comprises the amino acid sequence LRYGRQYS (SEQ ID NO:10).

In some aspects, a heterologous insertion (e.g., a CDRH3 or a target-binding fragment thereof or a BBB-binding domain) that binds to transferrin receptor comprises the amino acid sequence NVYAMTRNWWSDVY (SEQ ID NO:11), NVWHDLVWSVSTTDVY (SEQ ID NO:12), NVYARPRPDNLNWSDVY (SEQ ID NO: 13), HVETPYDSPELNWWDVY (SEQ ID NO:14), or NVYEL VEDTSAYEIGVDVY (SEQ ID NO:15).

In some aspects, a heterologous insertion (e.g., a CDRH3 or a target-binding fragment thereof or a BBB-binding domain) that binds to transferrin receptor comprises the amino acid sequence MLLRSDWYFDVW (SEQ ID NO:61), TGWGFDYW (SEQ ID NO: 16), TRGATALDYW (SEQ ID NO:17), NVYAMTRNWWSDVY (SEQ ID NO:18), NVWHDLVWSVSTTDVY (SEQ ID NO:19), NVYARPRPDNLNWSDVY (SEQ ID NO: 20), HVETPYDSPELNWWDVY (SEQ ID NO:21), or NVYELVEDTSAYEIGVDVY (SEQ ID NO:22).

In some aspects, a heterologous insertion (e.g., a CDRH3 or a target-binding fragment thereof or a BBB-binding domain) that binds to transferrin receptor comprises the amino acid sequence ARGLSGNYV (SEQ ID NO:23), ARGLSGNFV (SEQ ID NO:24), GLSGNYVMDY (SEQ ID NO:25), GLSGNYVVDY (SEQ ID NO:26), GLSGNFVMDF (SEQ ID NO:27) ARGTRA (SEQ ID NO:28) GTRAYHY (SEQ ID NO:29), GTYRAYHF (SEQ ID NO:30) ARYGYGNPATRY (SEQ ID NO:31), YGYGNPATRYFDV (SEQ ID NO: 32), TRGGYDSRAWF (SEQ ID NO:33), GGYDSRAWFAY (SEQ ID NO:34), GGYDSRAWFAH (SEQ ID NO:35), ARQGALYDGYYRGA (SEQ ID NO:36), QGALYDGYYRGAMDY (SEQ ID NO:37), ARRGYGYDGEF (SEQ ID NO:38), RGGYGYDGEFAY (SEQ ID NO:39), GLSGN (Y/F) V (M/V) D (Y/F) (SEQ ID NO:40), GTRAYH (Y/F) (SEQ ID NO:41) or (G/Q/R)G(Y/A/G)(D/L/Y)(S/Y/G)(R/D/Y)(A/G/D)(W/Y/G)(F/Y/E)(R/F/-)(G/-)(A/-)(M/-)(A/D)(Y/H) (SEQ ID NO:42).

In some aspects, a heterologous insertion (e.g., a CDRH3 or a target-binding fragment thereof or a BBB-binding domain) that binds to Basigin (Bsg) comprises the amino acid sequence VRLRGYFDY (SEQ ID NO:43), ARRDGSYYPYYWYFDL (SEQ ID NO: 44), ARTLINYSDYADYVMDA (SEQ ID NO:45), ARSLINYRNYGDYVMDA (SEQ ID NO:46), ARNGVYHNYWYFDF (SEQ ID NO:47), or AKEGGWFLRIYGMDY (SEQ ID NO:48).

In some aspects, a heterologous insertion (e.g., a CDRH3 or a target-binding fragment thereof or a BBB-binding domain) is capable of BBB transcytosis. The mechanism of transport across the blood brain barrier can be, e.g., receptor-mediated transport. See example in FIG. 1. The ability of a molecule comprising a heterologous insertion (e.g., a CDRH3 or a target-binding fragment thereof or a BBB-binding domain) to be transported across the BBB by receptor-mediated transport can be measured using an assay as described in Ribecco-Lutkiewicz M., et al., Sci. Rep 8 (1): 1873. doi: 10.1038/s41598-018-19522-8 (2018), which is herein incorporated by reference in its entirety. Such an assay can comprise exposing i-BECs (brain endothelial cells (BECs) generated from human amniotic fluid derived induced pluripotent stem cells (AF-iPSC)) to a labeled (fluorescent) version of the molecule comprising the heterologous insertion and acquiring images to determine if molecule was transported.

A heterologous insertion, as provided herein, can comprise at least 5 amino acids (e.g., can be 5-30 amino acids, 5-25 amino acids, 5-20 amino acids, 5-15 amino acids, or 5-14 amino acids). A heterologous insertion, as provided herein, can comprise at least 8 amino acids (e.g., can be 8-30 amino acids, 8-25 amino acids, 8-20 amino acids, 8-15 amino acids, or 8-14 amino acids). A heterologous insertion, as provided herein, can comprise at least 10 amino acids (e.g., can be 10-30 amino acids, 10-25 amino acids, 10-20 amino acids, 10-15 amino acids, or 10-14 amino acids). A heterologous insertion, as provided herein, can comprise up to 15 amino acids.

Also provided herein are libraries comprising heterologous insertions described herein.

### IV. Fusion Proteins and Antigen Binding Proteins Comprising Engineered Constant Domains

### A. Fusion Proteins

Provided herein are fusion proteins comprising (i) an engineered constant domain comprising a heterologous insertion in a constant domain and (ii) a heterologous polypeptide. The heterologous insertion can be, for example, a blood brain barrier (BBB)-binding domain. The heterologous insertion can be, for example, a heavy chain complementary determining region 3 (CDRH3) or a target-binding fragment thereof. In some aspects, the CDRH3 or target-binding fragment thereof binds to a BBB target.

In some aspects, the engineered constant domain in a fusion protein provided herein is N-terminal to the heterologous polypeptide. In some aspects, the engineered constant domain in a fusion protein provided herein is C-terminal to the heterologous polypeptide. In some aspects, a fusion protein provided herein comprises a heterologous polypeptide on both the N- and C-terminal sides of the engineered constant domain.

In some aspects, the constant domain is a CH3 domain. In some aspects, the fusion protein comprises (i) an Fc domain, wherein the Fc domain comprises a constant domain comprising a heterologous insertion and (ii) a heterologous polypeptide. In some aspects, the constant domain comprising a heterologous insertion is a CH3 domain.

In some aspects, the constant domain is a CH2 domain. In some aspects, the fusion protein comprises (i) an Fc domain, wherein the Fc domain comprises a constant domain comprising a heterologous insertion and (ii) a heterologous polypeptide. In some aspects, the constant domain comprising a heterologous insertion is a CH2 domain.

In some aspects, the constant domain is a CH1 domain. In some aspects, the fusion protein comprises (i) an Fc domain, wherein the Fc domain comprises a constant domain comprising a heterologous insertion and (ii) a heterologous polypeptide. In some aspects, the constant domain comprising a heterologous insertion is a CH1 domain.

In some aspects, the constant domain is a CL domain. In some aspects, the fusion protein comprises (i) an Fc domain, wherein the Fc domain comprises a constant domain comprising a heterologous insertion and (ii) a heterologous polypeptide. In some aspects, the constant domain comprising a heterologous insertion is a CL domain.

In some aspects, the heterologous polypeptide in a fusion protein provided herein is an enzyme (e.g., an enzyme for use in enzyme replacement therapy) or a catalytically active fragment thereof. In some aspects, the heterologous polypeptide in a fusion protein provided herein is a growth factor. In some aspects, the heterologous polypeptide in a fusion protein provided herein is a decoy receptor. In some aspects, the heterologous polypeptide in a fusion protein provided herein is progranulin or survival motor neuron protein (SMN).

In some aspects, a fusion protein provided herein does not comprise an antibody heavy chain variable region (VH). In some aspects, a fusion protein provided herein does not comprise an antibody light chain variable region (VL). In some aspects, a fusion protein provided herein does not comprise an antibody VH or VL. In some aspects, a fusion protein provided herein does not comprise an antigen-binding domain of an antibody.

In some aspects, a fusion protein provided herein further comprises a variable heavy chain (VH) or fragment thereof. In some aspects, a fusion protein provided herein further comprises a variable light chain (VL) or fragment thereof.

In some aspects, a fusion protein provided herein comprises an antigen-binding domain of an antibody. In some aspects, the antigen-binding domain binds to an antigen that is different than the BBB target bound by the heterologous insertion. Antigen-binding domains are described in detail in the next section.

### B. Antigen-Binding Proteins

According to some aspects provided herein, an antigen-binding protein can comprise an engineered constant domain and an antigen-binding domain, wherein the engineered constant domain comprises a heterologous insertion comprising a heavy chain complementary determining region 3 (CDRH3) or a target-binding fragment thereof. The CDRH3 or target-binding fragment can specifically bind to a blood brain barrier target.

According to some aspects provided herein, an antigen-binding protein can comprise an engineered constant domain and an antigen-binding domain, wherein the constant domain or comprises a heterologous insertion that specifically binds to a blood brain barrier protein target.

In some aspects, an antigen-binding protein (e.g., an antibody or antigen-binding fragment thereof) provided herein comprises a heavy chain and a light chain. With respect to the heavy chain, in some aspects, the heavy chain of an antigen-binding protein described herein can be an alpha (α), delta (δ), epsilon (ε), gamma (γ) or mu (μ) heavy chain. In some aspects, the heavy chain of an antigen-binding protein described can comprise a human alpha (α), delta (δ), epsilon (ε) gamma (γ) or mu (μ) heavy chain. In some aspects, the heavy chain of an antigen-binding protein described herein comprises a human gamma (γ) heavy chain constant region. In some aspects, the heavy chain of an antigen-binding protein described herein comprises the amino acid sequence of an IgG1 heavy chain constant region. In some aspects, the heavy chain of an antigen-binding protein described herein comprises the amino acid sequence of an IgG2 (e.g., IgG2a or IgG2b) heavy chain constant region. In some aspects, the heavy chain of an antigen-binding protein described herein comprises the amino acid sequence of an IgG4 heavy chain constant region. With respect to the light chain, in some aspects, the light chain of, an antigen-binding protein (e.g., an antibody or antigen-binding fragment thereof) described herein is a kappa light chain. In some aspects, the light chain of an antigen-binding protein (e.g., an antibody or antigen-binding fragment thereof) described herein is a lambda light chain. In some aspects, the light chain of an antigen-binding protein (e.g., an antibody or antigen-binding fragment thereof) described herein is a human kappa light chain or a human lambda light chain.

In some aspects, an antigen-binding protein (e.g., an antibody or antigen-binding fragment thereof) provided herein comprises constant regions comprising the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antigen-binding protein (e.g., an antibody or antigen-binding fragment thereof) provided herein comprises constant regions comprising the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Non-limiting examples of human constant region sequences have been described in e.g., U.S. Pat. No. 5,693,780 and Kabat E A et al., (1991) Sequences of Proteins of Immunological Interest, Fifth Edition, U.S. Department of Health and Human Services, NIH Publication No. 91-3242).

In some aspects an antigen-binding protein (e.g., an antibody or antigen-binding fragment thereof) provided herein comprises an engineered constant domain and an antigen-binding domain, wherein the engineered constant domain comprises a heterologous insertion comprising a CDRH3 or a target-binding fragment thereof. The antigen-binding domain can bind to an antigen. In some aspects, the CDRH3 or target-binding fragment thereof can bind to a BBB target. In some aspects, the antigen and the BBB target are not the same. In some aspects, the antigen-binding domain binds to a first protein and the CDRH3 binds to a second protein, wherein the first protein and the second protein are not the same.

In some aspects an antigen-binding protein (e.g., an antibody or antigen-binding fragment thereof) provided herein comprises an engineered constant domain and an antigen-binding domain, wherein the engineered constant domain comprises a heterologous insertion that binds to a BBB target. The antigen-binding domain can bind to an antigen. In some aspects, the antigen and the BBB target are not the same. In some aspects, the antigen-binding domain binds to a first protein and the heterologous insertion binds to a second protein, wherein the first protein and the second protein are not the same.

In some aspects provided herein, an antigen-binding domain (e.g., in a fusion protein, antibody or antigen-binding fragment thereof provided herein) specifically binds to a brain or CNS antigen. The brain or CNS antigen can be, for example, beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, TREM2, sortilin, CD33 or Siglec-3, Siglec-5, Siglec-7, Siglec-9, MS4A4A, MS4A6A, or TMEM106b.

In some aspects provided herein, an antigen-binding protein is an antibody, In some aspects, an antigen-binding protein is an antigen-binding fragment of an antibody, e.g., an scFv. In some aspects, an antigen-binding protein is homodimeric. In some aspects, an antigen-binding protein is heterodimeric.

As provided herein, the VH and VL or antigen-binding domain can bind to a CNS antigen or a brain antigen. Exemplary CNS antigen binding VH and VL sequences are provided below. Additional VH and VL and antigen-binding domain sequences are found in US2017/0224702, US 2018/0002433, US 2021/0236634, and US 2021/0238265, each of which is herein incorporated by reference in its entirety.

| CNS | | |
|---|---|---|
| Antigen | VH | VL |
| BACE1 | EVQLVESGGGLVQPGGSLRL | DIQMTQSPSSLSASVGDRVT |
| | SCAASGFTFSGYAIHWVRQA | ITCRASQDVSTAVAWYQQKP |
| | PGKGLEWVGWISPAGGSTDY | GKAPKLLIYSASFLYSGVPS |
| | ADSVKGRFTISADTSKNTAY | RFSGSGSGTDFTLTISSLQP |
| | LQMNSLRAEDTAVYYCARGP | EDFATYYCQQSYTTPPTFGQ |
| | FSPWVMDYWGQGTLVTVSS | GTKVEIKR |
| | (SEQ ID NO: 49) | (SEQ ID NO: 50) |
| BACE1 | EVQLVESGGGLVQPGGSLRL | DIQMTQSPSSLSASVGDRVT |
| | SCAASGFNFYYSSIHWVRQA | ITCRASQSVSSAVAWYQQKP |
| | PGKGLEWVASISPYSGYTSY | GKAPKLLIYSASSLYSGVPS |
| | ADSVKGRFTISADTSKNTAY | RFSGSGSGTDFTLTISSLQP |
| | LQMNSLRAEDTAVYYCARQP | EDFATYYCQQYSYSPFTFGQ |
| | THYYYYAKGYKAMDYWGQGT | GTKVEIKR |
| | LVTVSS | (SEQ ID NO: 52) |
| | (SEQ ID NO: 51) | |
| Alpha- | EVQLLESGGGLVQPGGSLRL | EIVLTQSPGTLSLSPGERAT |
| synuclein | SCAASGFTFSRFTMTWVRQA | LSCRASQSVSRSYLAWYQQK |
| | PGKGLEWVSAISGSGGGTSY | PGQAPRLLIYGASSRATGIP |
| | ADSVKGRLTVSRDNSKNTLY | DRFSGSGSGTDFTLTVSRLE |
| | LQMNSLRAEDTAVYYCAKNW | PEDFAVYYCQQYGSSPWTFG |
| | APFDYWGQGTLVTVSS | QGTKVEIK |
| | (SEQ ID NO: 53) | (SEQ ID NO: 54) |
| Alpha- | EVQLLESGGGLVQTGGSLRL | EIVLTQSPGTLSLSPGERAT |
| synuclein | SCAASGFTFSSYAMTWVRQA | LSCRASQSVSSSYLAWYQQK |
| | PGKGLEWVSAIRSNGDRTDY | PGQAPRLLIYGASSRATGIP |
| | ADSVKGRFTISRDNSQNTLY | DRFSGSGSGTDFTLTISRLE |
| | LQMNSLRAEDTAVYYCAKNW | PEDFAVYYCQQYGSSPWTFG |
| | APFDSWGQGTLVTVSS | QGTKVEIK |
| | (SEQ ID NO: 55) | (SEQ ID NO: 56) |
| Abeta | QVQLVQSGAEVKKPGSSVKV | DIVMTQTPLSLSVTPGQPAS |
| | SCKASGYDFTRYYINWVRQA | ISCKSSQSLLYSRGKTYLNW |
| | PGQGLEWMGWINPGSGNTKY | LLQKPGQSPQLLIYAVSKLD |
| | NEKFKGRVTITADESTSTAY | SGVPDRFSGSGSGTDFTLKI |
| | MELSSLRSEDTAVYYCAREG | SRVEAEDVGVYYCVQGTHYP |
| | ITVYWGQGTTVTVSS | FTFGQGTKLEIK |
| | (SEQ ID NO: 57) | (SEQ ID NO: 58) |
| Tau | EVQLVESGGGLVQPGGSLKL | SYELTQPPSVSVSPGQTARI |
| | SCAASGFNFNISAIHWVRQA | SCFGDTLPKQYTYWYQQKPG |
| | SGKGLEWVGRIRSKSHNYAT | QAPVLVIYKDTERPSGIPER |
| | LYAASLKGRFTLSRDDSRNT | FSGSSSGTTVTLTISGVQAE |
| | AYLQMSSLQTEDMAVYYCTV | DEADYYCLSADNSATWVFGG |
| | LGTKVTVL | SANYDTFDYWGQGTLVTVSS |
| | (SEQ ID NO: 60) | (SEQ ID NO: 59) |

In some aspects, an antigen-binding protein has a melting temperature (TM) of about 65° C. to about 75° C. In some aspects, an antigen-binding protein has a melting temperature (TM) of about 67° C. to about 73° C.

In some aspects, a fusion protein or antigen-binding protein (e.g., an antibody or antigen-binding fragment thereof) provided herein has a TM that is similar to the TM of, a fusion protein or antigen-binding protein (e.g., an antibody or antigen-binding fragment thereof) that is identical except that it lacks the heterologous insertion.

In some aspects, a fusion protein or antigen-binding protein (e.g., an antibody or antigen-binding fragment thereof) provided herein binds to a blood brain barrier target with an affinity of about 1 nM to about 1000 nM. In some aspects, a fusion protein or antigen-binding protein (e.g., an antibody or antigen-binding fragment thereof) provided herein binds to a blood brain barrier target with an affinity of about 1 nM to about 500 nM. In some aspects, a fusion protein or antigen-binding protein (e.g., an antibody or antigen-binding fragment thereof) provided herein binds to a blood brain barrier target with an affinity of about 1 nM to about 250 nM. In some aspects, a fusion protein or antigen-binding protein (e.g., an antibody or antigen-binding fragment thereof) provided herein binds to a blood brain barrier target with an affinity of about 1 nM to about 100 nM. In some aspects, a fusion protein or antigen-binding protein (e.g., an antibody or antigen-binding fragment thereof) provided herein binds to a blood brain barrier target with an affinity of about 1 nM to about 50 nM.

In some aspects, a fusion protein or antigen-binding protein (e.g., an antibody or antigen-binding fragment thereof) provided herein binds to neonatal Fc receptor (FcRn). In some aspects, a fusion protein or antigen-binding protein (e.g., an antibody or antigen-binding fragment thereof) provided herein binds to FcRn with a monomeric affinity of ≤ 1000 nM.

In some aspects, a fusion protein or antigen-binding protein (e.g., an antibody or antigen-binding fragment thereof) provided herein that binds to transferrin receptor does not block iron transport. In some aspects, a fusion protein or antigen-binding protein (e.g., an antibody or antigen-binding fragment thereof) provided herein that binds to transferrin receptor does not decrease iron transport by more than 50% (e.g., as compared to iron transport in the absence of the fusion protein or antigen-binding protein). In some aspects, a fusion protein or antigen-binding protein (e.g., an antibody or antigen-binding fragment thereof) provided herein that binds to transferrin receptor does not decrease iron transport by more than 25% (e.g., as compared to iron transport in the absence of the fusion protein or antigen-binding protein). In some aspects, a fusion protein or antigen-binding protein (e.g., an antibody or antigen-binding fragment thereof) provided herein that binds to transferrin receptor does not decrease iron transport by more than 10% (e.g., as compared to iron transport in the absence of the fusion protein or antigen-binding protein). In some aspects, a fusion protein or antigen-binding protein (e.g., an antibody or antigen-binding fragment thereof) provided herein that binds to transferrin receptor does not decrease iron transport by more than 5% (e.g., as compared to iron transport in the absence of the fusion protein or antigen-binding protein).

In some aspects, a fusion protein or antigen-binding protein (e.g., an antibody or antigen-binding fragment thereof) provided herein that binds to transferrin receptor does not reduce the level of TfR-expressing peripheral cells by more than 15% (e.g., as compared to iron transport in the absence of the fusion protein or antigen-binding protein). In some aspects, a fusion protein or antigen-binding protein (e.g., an antibody or antigen-binding fragment thereof) provided herein that binds to transferrin receptor does not reduce the level of TfR-expressing peripheral cells by more than 10% (e.g., as compared to iron transport in the absence of the fusion protein or antigen-binding protein). In some aspects, a fusion protein or antigen-binding protein (e.g., an antibody or antigen-binding fragment thereof) provided herein that binds to transferrin receptor does not reduce the level of TfR-expressing peripheral cells by more than 5% (e.g., as compared to iron transport in the absence of the fusion protein or antigen-binding protein).

In some aspects, a fusion protein or antigen-binding protein (e.g., an antibody or antigen-binding fragment thereof) provided herein comprises a dimer. In some aspects, the dimer comprises two constant domains or fragments thereof. In some aspects, each constant domain of the dimer comprises a heterologous insertion. The heterologous insertion in the constant domains can be the same or can be different. In some aspects, a fusion protein or antigen-binding protein (e.g., an antibody or antigen-binding fragment thereof) provided herein comprises two constant domains or fragments thereof, wherein only one constant domain comprise a heterologous insertion comprising a BBB target-binding domain and/or a CDRH3 or target-binding domain thereof.

In some aspects, a fusion protein or antigen-binding protein (e.g., an antibody or antigen-binding fragment thereof) provided herein comprises one or more linkers. For example, a fusion protein or antigen-binding protein (e.g., an antibody or antigen-binding fragment thereof) can comprise a first linker at the N-terminus of the heterologous insertion. A fusion protein or antigen-binding protein (e.g., an antibody or antigen-binding fragment thereof) can also comprise a second linker at the C-terminus of the heterologous insertion. In some aspect, a fusion protein or antigen-binding protein (e.g., an antibody or antigen-binding fragment thereof) comprises a first linker at the N-terminus of the heterologous insertion and/or a second linker at the C-terminus of the heterologous insertion, wherein the first and second linkers are capable of forming a bridge (e.g. a cysteine bridge) with each other.

In some aspects, a linker (e.g., a linker at the N- and/or C-terminal of the heterologous insertion) comprises the amino acid sequence GGG. In some aspects, a linker (e.g., an N-terminal linker and/or a C-terminal linker) comprises the amino acid sequence CGG. In some aspects, a linker (e.g., a linker at the N- and/or C-terminal of the heterologous insertion) comprises the amino acid sequence GGC. In some aspects, a linker at the N-terminal of the heterologous insertion comprises the amino acid sequence GGC and a linker at the C-terminal of the heterologous insertion comprises the amino acid sequence CGG. In some aspects, a linker at the N-terminal of the heterologous insertion comprises the amino acid sequence CGG and the a linker at the C-terminal of the heterologous insertion comprises the amino acid sequence GGC.

In some aspect, a fusion protein or antigen-binding protein (e.g., an antibody or antigen-binding fragment thereof) comprises a linker at the N-terminal of the heterologous insertion and a linker at the C-terminal of the heterologous insertion, wherein the linkers are capable of forming a salt bridge with each other, e.g., wherein one linker is positively charged and the other linker is negatively charged.

In some aspects, a linker (e.g., a linker at the N- and/or C-terminal of the heterologous insertion) is a proline linker, e.g., a polyproline linker, or a proline-rich linker.

In some aspects, a linker (e.g., a linker at the N- and/or C-terminal of the heterologous insertion) is a glycine-serine linker, e.g., comprising GGS.

In some aspects a fusion protein or antigen-binding protein (e.g., an antibody or antigen-binding fragment thereof) comprising a constant domain comprising a heterologous insertion provided herein is capable of BBB transcytosis. The mechanism of transport across the blood brain barrier can be, e.g., receptor-mediated transport.

In some aspects, at least 0.5% of an fusion protein or antigen-binding protein (e.g., an antibody or antigen-binding fragment thereof) comprising a constant domain comprising a heterologous insertion as provided herein accumulates in a vessel-depleted brain fraction of mice injected intravenously with 5 mg/kg of the fusion protein or antigen-binding protein.

In some aspects, a fusion protein or antigen-binding protein (e.g., an antibody or antigen-binding fragment thereof) comprising a constant domain comprising a heterologous insertion provided herein has a 3 to 100-fold increase in accumulation in vessel-depleted brain fraction of mice injected intravenously with the fusion protein or antigen-binding protein as compared to a fusion protein or antigen-binding protein that is identical except that it lacks the heterologous insertion.

In some aspects, a fusion protein or antigen-binding protein (e.g., an antibody or antigen-binding fragment thereof) provided herein comprises a detectable label.

Also provided herein are libraries comprising constant domains, polypeptides, fusion proteins, or antigen-binding proteins (e.g., an antibody or antigen-binding fragment thereof) described herein.

### V. Polynucleotides and Methods of Making Engineered Constant Domains and Fusion Proteins Comprising the Same

In certain aspects, provided herein are polynucleotides comprising a nucleotide sequence encoding an engineered constant domain, fusion protein, or antigen-binding protein (e.g., an antibody or antigen-binding fragment thereof) or a domain thereof as described herein, and vectors, e.g., vectors comprising such polynucleotides for recombinant expression in host cells (e.g., E. coli and mammalian cells).

Also provided herein are polynucleotides comprising a nucleotide sequence encoding an engineered constant domain, polypeptide, fusion protein, or antigen-binding protein (e.g., an antibody or antigen-binding fragment thereof) or a domain thereof described herein that are optimized, e.g., by codon/RNA optimization, replacement with heterologous signal sequences, and/or elimination of mRNA instability elements. Methods to generate optimized nucleic acids for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

A polynucleotide comprising a nucleotide sequence encoding an engineered constant domain, fusion protein, or antigen-binding protein (e.g., an antibody or antigen-binding fragment thereof) or a domain thereof described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising, e.g., the sequence encoding the light chain and/or heavy chain of an antibody or antigen-binding fragment thereof, wherein the light chain and/or heavy chain comprises a heterologous insertion in a constant domain. Such PCR amplification methods can be used to obtain nucleic acids comprising the sequence encoding light chain region and/or the heavy chain region of an antibody or antigen-binding fragment thereof. The amplified nucleic acids can be cloned into vectors for expression in host cells and for further cloning, for example, to generate an engineered constant domain, fusion protein, or antigen-binding protein (e.g., an antibody or antigen-binding fragment thereof) or a domain thereof described herein.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure.

Also provided herein are libraries comprising polynucleotides described herein.

Methods of making polynucleotides are also provided herein. For example, provided herein are methods of making a polynucleotide comprising a nucleic acid sequence encoding an engineered constant domain comprising a heterologous insertion. In some aspects, such a method comprises (a) providing a first nucleic acid sequence encoding a constant domain and (b) inserting a heterologous nucleic acid sequence into the region of the first nucleic acid sequence encoding the constant domain to produce the polynucleotide. In some aspects, the heterologous nucleic acid sequence encodes a CDRH3 or a target-binding fragment thereof. In some aspects, the heterologous nucleic acid sequence encodes an amino acid sequence that binds to a blood brain barrier target. In some aspects, the method further comprises linking the nucleic acid sequence encoding a constant domain comprising a heterologous insertion to nucleic acid sequence encoding a heterologous protein, e.g., an enzyme useful for enzyme replacement therapy, a growth factor, a receptor decoy, or an antibody or fragment thereof. The resulting polynucleotide can comprise a nucleic acid sequence encoding an fusion protein comprising a constant domain comprising a heterologous insertion. Polynucleotides produced by such methods are also provided herein.

Also provided herein are methods for making a polynucleotide comprising a nucleic acid sequence encoding an fusion protein comprising a constant domain comprising a heterologous insertion. In some aspects, such a method comprises (a) providing a first nucleic acid sequence encoding a fusion protein comprising a constant domain and (b) inserting a heterologous nucleic acid sequence into the region of the first nucleic acid sequence encoding the constant domain to produce the polynucleotide. In some aspects, the heterologous nucleic acid sequence encodes a CDRH3 or a target-binding fragment thereof. In some aspects, the heterologous nucleic acid sequence encodes an amino acid sequence that binds to a blood brain barrier target. Polynucleotides produced by such methods are also provided herein.

Also provided herein are methods for making a polynucleotide comprising a nucleic acid sequence encoding an antigen-binding protein comprising a constant domain comprising a heterologous insertion. In some aspects, such a method comprises (a) providing a first nucleic acid sequence encoding an antigen-binding protein comprising a constant domain and (b) inserting a heterologous nucleic acid sequence into the region of the first nucleic acid sequence encoding the constant domain to produce the polynucleotide. In some aspects, the heterologous nucleic acid sequence encodes a CDRH3 or a target-binding fragment thereof. In some aspects, the heterologous nucleic acid sequence encodes an amino acid sequence that binds to a blood brain barrier target. Polynucleotides produced by such methods are also provided herein.

Also provided herein are methods of making a polynucleotide encoding a polypeptide comprising a constant domain that comprises a heterologous insertion that confers target binding specificity. In some aspects, the method comprises providing a first nucleic acid sequence encoding a CDRH3 derived from an antibody or antigen binding fragment that binds to a target of interest, inserting the first nucleic acid sequence into a second nucleic acid sequence encoding a polypeptide comprising a constant domain, such that the resulting nucleic acid encodes a polypeptide comprising a constant domain that comprises a heterologous insertion that confers target binding specificity.

In certain aspects, provided herein are vectors (e.g., expression vectors) comprising polynucleotides comprising nucleotide sequences encoding an engineered constant domain, polypeptide comprising an engineered constant domain, fusion protein, or antigen-binding protein (e.g., an antibody or antigen-binding fragment thereof) or a domain thereof as described herein for recombinant expression in a host cell, e.g., in a mammalian host cell. A vector for the production of the engineered constant domain, polypeptide comprising an engineered constant domain, fusion protein, or antigen-binding protein (e.g., an antibody or antigen-binding fragment thereof) or a domain thereof can be produced, e.g., by recombinant DNA technology using techniques well known in the art. These methods include, for example, in vitro recombinant DNA techniques, synthetic techniques, and in vivo genetic recombination. Also provided are replicable vectors comprising a nucleotide sequence encoding an engineered constant domain, polypeptide, fusion protein, or antigen-binding protein (e.g., an antibody or antigen-binding fragment thereof) or a domain thereof, operably linked to a promoter. Such vectors can, for example, include the nucleotide sequence encoding the constant region of an antibody or antigen-binding fragment thereof (see, e.g., International Publication Nos. WO 86/05807 and WO 89/01036; and U.S. Pat. No. 5,122,464), and variable domains of the antibody or antigen-binding fragment thereof can be cloned into such a vector for expression of the entire heavy, the entire light chain, or both the entire heavy and light chains.

In certain aspects, provided herein are expression systems comprising polynucleotides comprising nucleotide sequences encoding an engineered constant domain, polypeptide, fusion protein, or antigen-binding protein (e.g., an antibody or antigen-binding fragment thereof) or a domain thereof as described herein. An expression system can be included on a vector. An expression system can also be integrated into a host cell chromosome. In some aspects, an expression system is a cell free expression system. In some aspects, an expressions system comprises a host cell comprising a polynucleotide and/or vector provided herein.

Accordingly, also provided herein are cells, e.g. host cells, comprising polynucleotides and/or vectors for recombinantly expressing an engineered constant domain, polypeptide, fusion protein, or antigen-binding protein (e.g., an antibody or antigen-binding fragment thereof) or a domain thereof as described herein. In some aspects, for the expression of double-chained antigen-binding proteins, vectors encoding both the heavy and light chains, individually, can be co-expressed in the host cell for expression of the entire immunoglobulin. In some aspects, a host cell contains two different vectors, a first vector comprising a polynucleotide encoding a heavy chain of an antigen-binding protein described herein, and a second vector comprising a polynucleotide encoding a light chain of the an antigen-binding protein. In some aspects, a first host cell comprises a first vector comprising a polynucleotide encoding a heavy chain, and a second host cell comprises a second vector comprising a polynucleotide encoding a light chain. In some aspects, provided herein is a population of host cells comprising such first host cell and such second host cell.

In some aspects, provided herein is a population of vectors comprising a first vector comprising a polynucleotide encoding a light chain of an antigen-binding protein described herein, and a second vector comprising a polynucleotide encoding a heavy chain of the an antigen-binding protein. Alternatively, a single vector can be used which encodes, and is capable of expressing, both heavy and light chain polypeptides.

In some aspects, provided herein are methods for producing an engineered constant domain, polypeptide, fusion protein, or antigen-binding protein (e.g., an antibody or antigen-binding fragment thereof) or a domain thereof as described herein, comprising expressing such a constant domain, polypeptide, fusion protein, or antigen-binding protein (e.g., an antibody or antigen-binding fragment thereof) or a domain in a host cell. An expression vector can be transferred to a cell (e.g., host cell) by conventional techniques, and the resulting cells can then be cultured by conventional techniques to produce an engineered constant domain, polypeptide, fusion protein, or antigen-binding protein (e.g., an antibody or antigen-binding fragment thereof) or a domain thereof as described herein.

A variety of host-expression vector systems can be utilized to express an engineered constant domain, polypeptide, fusion protein, or antigen-binding protein (e.g., an antibody or antigen-binding fragment thereof) as described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an engineered constant domain, polypeptide, fusion protein, or antigen-binding protein (e.g., an antibody or antigen-binding fragment thereof) as described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing an engineered constant domain, polypeptide, fusion protein, or antigen-binding protein (e.g., an antibody or antigen-binding fragment thereof) as described herein are CHO cells, for example CHO cells from the CHO GS System™ (Lonza). In some aspects, cells for expressing an engineered constant domain, polypeptide, fusion protein, or antigen-binding protein (e.g., an antibody or antigen-binding fragment thereof) as described herein are human cells, e.g., human cell lines. In some aspects, a mammalian expression vector is pOptiVEC™ or pcDNA3.3. In some aspects, bacterial cells, such as Escherichia coli, or eukaryotic cells (e.g., mammalian cells) are used for the expression of an engineered constant domain, polypeptide, fusion protein, or antigen-binding protein (e.g., an antibody or antigen-binding fragment thereof) as described herein. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In some aspects, an engineered constant domain, polypeptide, fusion protein, or antigen-binding protein (e.g., an antibody or antigen-binding fragment thereof) as described herein are produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Also provided herein are methods for adding a binding specificity to a protein comprising an engineered constant domain. In some aspects, such a method comprises (a) providing a first nucleic acid sequence encoding an a protein comprising a constant domain, (b) inserting a heterologous nucleic acid sequence into the first nucleic acid sequence to produce a modified polynucleotide, and (c) expressing the modified polynucleotide in an expression system. In some aspects, the heterologous nucleic acid sequence encodes a CDRH3 or a target-binding fragment thereof. In some aspects, the heterologous nucleic acid sequence encodes an amino acid sequence that binds to a blood brain barrier target, optionally wherein the amino acid sequence that binds to a blood brain barrier target is a CDRH3 or a target-binding fragment thereof. In some aspects, the protein comprising the constant domain is an antigen-binding protein. In some aspects, the antigen-binding protein is an antibody or antigen-binding fragment thereof.

In some aspects, a heterologous nucleic acid in a polynucleotide, vector, or expression system provided herein can be mutagenized. The mutagenizing can produce a library of polynucleotides, vectors, or expression systems. Libraries of polynucleotides, vectors, or expression systems produced by such methods are also provided herein.

Once an engineered constant domain, polypeptide, fusion protein, or antigen-binding protein (e.g., an antibody or antigen-binding fragment thereof) as described herein has been produced by recombinant expression, it can be purified by any method known in the art for purification, for example, by chromatography (e.g., ion exchange, affinity, particularly by affinity for the specific antigen after Protein A, and sizing column chromatography), centrifugation, differential solubility, or by any other standard technique for the purification of proteins. Further, the engineered constant domain, polypeptide, fusion protein, or antigen-binding protein (e.g., an antibody or antigen-binding fragment thereof) described herein can be fused to heterologous polypeptide sequences to facilitate purification.

In some aspects, an engineered constant domain, polypeptide, fusion protein, or antigen-binding protein (e.g., an antibody or antigen-binding fragment thereof) described herein is isolated or purified. Generally, an isolated engineered constant domain, polypeptide, fusion protein, or antigen-binding protein is one that is substantially free of other proteins. For example, in some aspects, a preparation of an engineered constant domain, polypeptide, fusion protein, or antigen-binding protein (e.g., an antibody or antigen-binding fragment thereof) described herein is substantially free of cellular material and/or chemical precursors.

In some aspects, a method provided herein further comprises screening a protein or a library of proteins produced by a method provided herein for the ability to bind to a blood brain barrier protein. In some aspects, a method provided herein further comprises screening a protein or a library of proteins produced by a method provided herein for the ability to cross the blood brain barrier.

Also provided herein are methods for producing a protein with the ability to cross a blood brain barrier. See FIG. 5. In some aspects, such a method comprises (i) screening a library of proteins containing CDRH3 domains (e.g., proteins containing CDRH3 domains with known sequences) for the ability to bind a blood brain barrier target, (ii) selecting a protein from the library of proteins and identifying the CDRH3 domain of the selected protein, (iii) inserting a nucleic acid sequence encoding the CDRH3 domain or a fragment thereof of the selected protein into a nucleic acid sequence encoding a protein comprising a constant domain to produce a polynucleotide, wherein the insertion is within the region of the nucleic acid encoding the constant domain, and (iv) expressing the polynucleotide in a host cell. In some aspects, such a method comprises (i) inserting a nucleic acid sequence encoding a CDRH3 domain or a target-binding fragment thereof into a nucleic acid sequence encoding a protein comprising a constant domain to produce a polynucleotide, wherein the insertion is within the region of the nucleic acid encoding the constant domain, (ii) expressing the polynucleotide in a host cell to produce a protein, and (iii) screening the protein for the ability to bind a blood brain barrier target and/or cross the blood brain barrier. The protein can be, for example, an antigen-binding protein. The antigen-binding protein can be, for example, an antibody or antigen-binding fragment thereof. In some aspects, the resulting protein (e.g., antigen-binding protein) has a 3-100-fold increase in accumulation in vessel-depleted brain fraction of mice injected intravenously with the protein as compared to a protein that is identical except that it lacks the CDRH3 domain or a target-binding fragment thereof. In some aspects, the resulting protein (e.g., antigen-binding protein) has a 3-100-fold increase in accumulation in the brain of an individual administered the resulting protein.

Also provided herein are methods of engineering a protein capable of crossing the blood brain barrier. In some aspects, such a method comprises (i) providing a first nucleic acid encoding a protein, wherein the protein comprises a constant domain, (ii) inserting a second nucleic acid encoding a CDRH3 domain or fragment thereof into the portion of the first nucleic acid that encodes the constant domain to produce a modified nucleic acid encoding a modified protein, (iii) introducing the modified nucleic acid into an expression system, (iv) expressing the modified protein, and (v) screening the modified protein for binding to a blood brain barrier target and/or for crossing the blood brain barrier. The protein can be, for example, an antigen-binding protein. The antigen-binding protein can be, for example, an antibody or antigen-binding fragment thereof. In some aspects, the resulting protein (e.g., antigen-binding protein) has a 3-100-fold increase in accumulation in vessel-depleted brain fraction of mice injected intravenously with the protein as compared to a protein that is identical except that it lacks the CDRH3 domain or a target-binding fragment thereof.

In some aspects, of the methods provided herein, a library contains sequences encoding CDRH3 regions from antibodies or antigen-binding fragments thereof known to bind to a blood brain barrier target. In some aspects, the library contains determined sequences (e.g., determined by sequencing) encoding CDRH3 regions from antibodies or antigen-binding fragments thereof known to bind to a blood brain barrier target. In some aspects, of the methods provided herein, a library contains sequences encoding CDRH3 regions from antibodies or antigen-binding fragments thereof not known to bind to a blood brain barrier target.

Also provided herein are libraries of proteins (e.g., antigen-binding proteins or fusion proteins) produced according to a method provided herein.

### VI. Pharmaceutical Compositions

Provided herein are compositions comprising an engineered constant domain, polypeptide, fusion protein, or antigen-binding protein (e.g., an antibody or antigen-binding fragment thereof) as described herein. In some aspects, the engineered constant domain, polypeptide, fusion protein, or antigen-binding protein (e.g., an antibody or antigen-binding fragment thereof) having the desired degree of purity is present in a formulation comprising, e.g., a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed. Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an engineered constant domain, polypeptide, fusion protein, or antigen-binding protein (e.g., an antibody or antigen-binding fragment thereof) as described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Pharmaceutical compositions described herein are, in some aspects, for use as a medicament. The compositions to be used for in vivo administration can be sterile. This is readily accomplished by filtration through, e.g., sterile filtration membranes.

A pharmaceutical composition described herein can be used to exert a biological effect(s) in vivo or in vitro. For example, a pharmaceutical composition described herein can be used to cross a blood brain barrier, e.g., in a subject.

In some aspects, a pharmaceutical composition provided herein is used to treat diseases or conditions such as a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some aspects, a pharmaceutical composition provided herein is used to treat diseases or conditions such as Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury. In some aspects, a pharmaceutical composition provided herein is used to treat frontotemporal dementia.

In some aspects, a pharmaceutical composition provided herein is formulated for intravenous administration. In some aspects, a pharmaceutical composition provided herein is formulated for subcutaneous administration.

### VII. Methods of Using Engineered Constant Domains and Fusion Proteins Comprising the Same

Fusion proteins and antigen-binding proteins comprising a constant domain comprising a heterologous insert (e.g., a CDRH3 or target-binding fragment thereof and/or a blood brain barrier-binding insert) as provided herein can advantageously be transported across a blood brain barrier. Accordingly, provided herein are method of administering or transporting a fusion protein or an antigen-binding protein across the blood brain barrier of a subject comprising administering a fusion protein or antigen-binding protein provided herein to the subject.

In view of the ability of fusion proteins and antigen-binding proteins comprising a constant domain comprising a heterologous insert (e.g., a CDRH3 or target-binding fragment thereof and/or a blood brain barrier-binding insert) as provided herein to be transported across a blood brain barrier, they can be used to treat a neurological disease or disorder. In some aspects, a method of treating a neurological disease or disorder in a subject comprises administering a fusion protein or antigen-binding protein provided herein to the subject. The neurological disease or disorder can be, for example, a neuropathy disorder, a neurodegenerative disease, cancer, an ocular disease disorder, a seizure disorder, a lysosomal storage disease, amyloidosis, a viral or microbial disease, ischemia, a behavioral disorder, or CNS inflammation. The neurological disease or disorder can be, for example, Alzheimer's disease (AD), stroke, dementia, muscular dystrophy (MD), multiple sclerosis (MS), amyotrophic lateral sclerosis (ALS), limbic-predominant age-related TDP-43 encephalopathy (LATE), cystic fibrosis, Angelman's syndrome, Liddle syndrome, Parkinson's disease, Pick's disease, Paget's disease, cancer, or traumatic brain injury. In some aspects, the neurological disease or disorder is dementia. In some aspects, the neurological disease or disorder is frontotemporal dementia.

Antigen-binding proteins (e.g., antibodies and antigen-binding fragments thereof) comprising a constant domain comprising a heterologous insert (e.g., a CDRH3 or target-binding fragment thereof and/or a blood brain barrier-binding insert) as provided herein can be used to detect an antigen (e.g., a CNS antigen or a brain antigen). Antigen-binding proteins for such purposes can be labeled. Exemplary labels include, for example, radioisotopes (e.g., 64CU) and fluorescent labels. Accordingly, methods of detecting an antigen using an antigen-binding protein provided herein are provided. In some aspects, a method of detecting an antigen in the CNS (e.g., brain) of a subject comprises administering an antigen-binding protein provided herein to the subject and detecting the binding of the antigen-binding protein to the antigen in the CNS (e.g., brain). Such methods can further comprise, e.g., performing Positron emission tomography (PET) imaging on the subject.

Antigen-binding proteins (e.g., antibodies and antigen-binding fragments thereof) comprising a constant domain comprising a heterologous insert (e.g., a CDRH3 or target-binding fragment thereof and/or a blood brain barrier-binding insert) as provided herein can be used for prognostic, diagnostic, monitoring, and/or screening applications, including in vivo applications well known and standard to the skilled artisan and based on the present description. In some aspects, provided herein is an antigen-binding protein provided herein for use as a diagnostic. In some aspects, the antigen-binding protein comprises a detectable label.

### EXAMPLES

### Example 1: CDRH3 Grafting in a Constant Domain to Generate LP1 Antibody

This example describes CDRH3 grafting into the CH3 domain of a human IgG1 antibody heavy chain constant region, with the CDRH3 replacing N384 and G385 (EU numbering of the human IgG1 heavy chain) of the antibody.

CDRH3s were identified from anti-transferrin receptor (TfR) antibodies and antigen-binding fragments originating from several sources, including a mouse scFv library, a fully human scFv library, and mouse hybridoma derived full-length antibodies. CDRH3s of the antibodies and antigen-binding fragments were determined using the IMGT standard. One scFv from the mouse scFv library had the following amino acid sequence:

(SEQ ID NO: 1)
DYKDIVMTQSHKFMSTSVGDRVSITCKASQDVGTAVAWYQQKPGQ
SPKLLIYWASTRHTGVPDRFTGSGSGTDFTLTISNVQSEDLADYF
CQQYSSYLTFGAGTKLELKRGGGGSGGGGSGGGGSGGGGSEVMLV
ESGGDLVKPGGSLKLSCAASGFTFSSYGMSWVRQTPDKRLEWVAT
ISSGGSYTYYPDSVKGRFTISRDNAKNTLYLQMSSLKSEDTAMYY
CALLRSDWYFDVWGAGTTVTVSS

The CDRH3 of the scFv of SEQ ID NO:1 was identified as ALLRSDWYFDVW (SEQ ID NO:2). This CDRH3 sequence (with the “A” substituted with “M”) was inserted into a sequence comprising a native (wild type or “WT”) hIgG1 constant sequence (SEQ ID NO: 3) to produce a grafted protein comprising a WT hIgG1 constant sequence comprising a heterologous CDRH3 insertion in the CH3 domain (SEQ ID NO:4).

(SEQ ID NO: 3)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
(SEQ ID NO: 4)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESMLLR
SDWYFDVWQPENNYKTTPPVLDSDGSFFLYSKLTVDKSRWQQGNV
FSCSVMHEALHNHYTQKSLSLSPGK

DNA encoding for SEQ ID NO:4 was generated and cloned into an expression plasmid encoding for the heavy chain of an isotype control antibody with variable domains lacking binding to any mammalian protein. The sequence encoding SEQ ID NO:4 replaced the corresponding sequence in the heavy chain-encoding plasmid. A full length hIgG1 antibody called “LP1,” in which the modified heavy chain as described above was paired with a human IgG1 light chain, was produced recombinantly via transfection into Expi293 cells and purified using MabSelect Sure resin.

### Example 2: Biolayer Interferometry (BLI) Measurement of Binding of CDRH3-Grafted Antibody LP1 to CDRH3-Target Protein

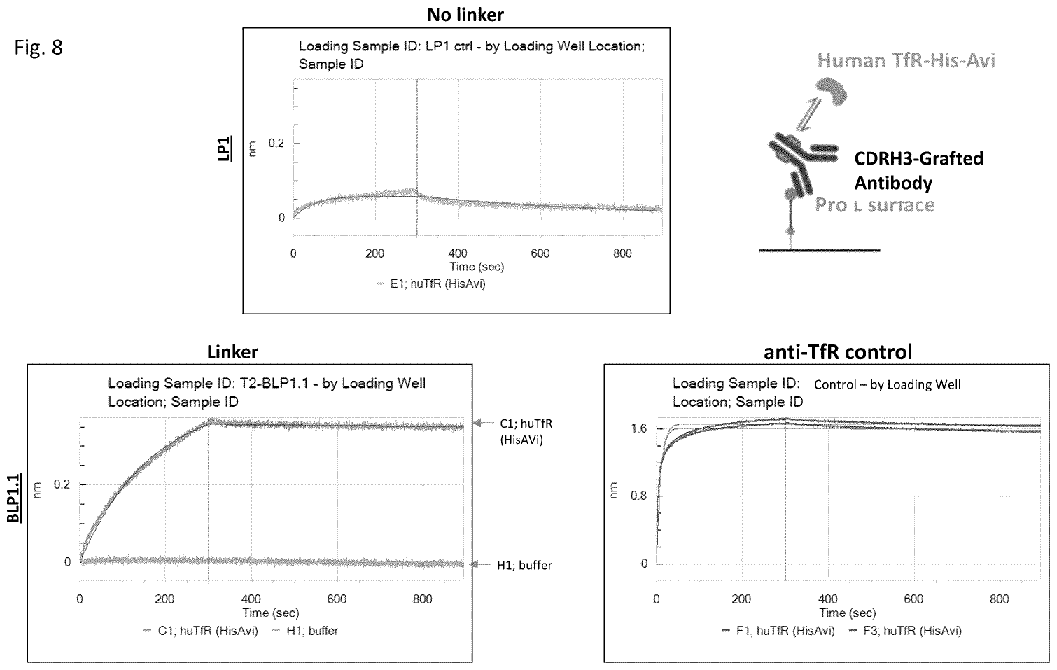

In order to measure the binding of CDRH3-grafted antibodies to the CDRH3-target protein (human transferrin receptor (huTfR), biolayer interferometry (BLI) assays were performed. A diagram of the assay is shown in FIG. 8. Antibodies were diluted to 5.0 μg/mL and loaded onto Pro L tips (Sartorius, Cat. No. 18-5085) for three minutes at 600 rotations per minute (rpm), followed by a wash step for 60 seconds at 1000 rpm in running buffer (1×PBS, 0.5 mg/mL BSA, 0.05% Tween 20, pH 7.4). Antibody-loaded tips were associated to 1.25 μM of antigen (recombinant huTfR (extracellular domain) fused to an AVI-His tag (“huTfR-HisAvi”)) solution, for five minutes at 1000 rpm, followed by 10 minutes of dissociation in running buffer. Tip regeneration was performed by dipping tips twice into 1/200 (v/v) phosphoric acid solution, for 15 seconds each, at 1000 rpm, with 15 seconds wash step in running buffer, in after each regeneration injection. Antibody and antigen solutions were diluted in running buffer (1×PBS, 0.5 mg/mL BSA, 0.05% Tween 20, pH 7.4). A sample well with positive control (anti-TfR) antibody was included in each “loading” column to monitor instrument variability

Raw data from active surfaces (antibody present) was double-referenced with reference tips (antibody absent) and buffer injections over active surfaces. The referenced sensogram data was fitted to a 1:1 kinetic binding model using ForteBio's Data Analysis Software (version 9.0.0.14). Raw data was collected with an OctetRed instrument and Octet Data Acquisition software (version 9.0.0.49). The results are shown in FIG. 8 (top graph (LP1) and lower right graph (positive control)). The results show that LP1 binds with substantially weaker affinity than positive control.

### Example 3: Insertion Site and Linker Engineering of LP1 to Generate Modified Antibodies

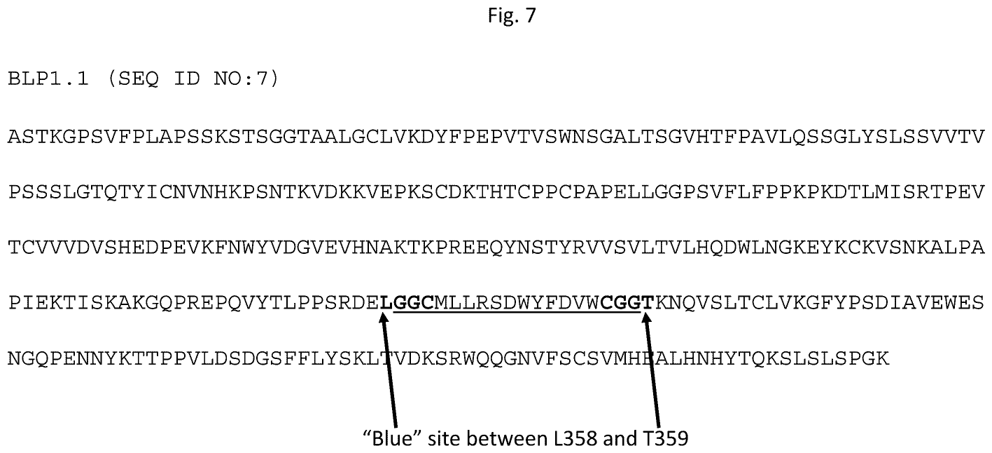

In order to enhance the binding affinity of the hIgG1 LP1 constant region to TfR, a small library of variants was made to test inserting the CDRH3 at different sites and with different linkers. Two insertion sites were tested in this round: the “Gold” or “G” site-between N384 and G385; and the “Blue” or “B” site-between L358 and T359 (EU numbering of the hIgG1 heavy chain). For each site, two linkers were tested, either-GGG-CDRH3-GGG- or -GGC-CDRH3-CGG-, resulting in a library of 4 sequences. The modified versions of LP1 are referred to as GLP1.1 (SEQ ID NO. 5), GLP1.2 (SEQ ID NO. 6), BLP1.1 (SEQ ID NO. 7) and BLP1.2 (SEQ ID NO. 8). See FIG. 7 for “Blue” site in BLP1.1.

(SEQ ID NO: 5)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGG
CMLLRSDWYFDVWCGGGQPENNYKTTPPVLDSDGSFFLYSKLTVD
KSRWQQGNVFSCSVMHEALHNHYTQKSLSLSPGK
(SEQ ID NO: 6)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGG
GMLLRSDWYFDVWGGGGQPENNYKTTPPVLDSDGSFFLYSKLTVD
KSRWQQGNVFSCSVMHEALHNHYTQKSLSLSPGK
(SEQ ID NO: 7)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELGGCMLLRSDWYFDVWCGGTKNQVSLTCLV
KGFYPSDIAVEWESNGQPENNYKTTPPVLDSDGSFFLYSKLTVDK
SRWQQGNVFSCSVMHEALHNHYTQKSLSLSPGK
(SEQ ID NO: 8)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELGGGMLLRSDWYFDVWGGGTKNQVSLTCLV
KGFYPSDIAVEWESNGQPENNYKTTPPVLDSDGSFFLYSKLTVD
KSRWQQGNVFSCSVMHEALHNHYTQKSLSLSPGK

DNA encoding for SEQ ID NOs: 5-8 were generated and cloned into an expression plasmid encoding for the heavy chain of an isotype control antibody with variable domains that lack binding to any mammalian protein. The sequences encoding SEQ ID NOs: 5-8 replaced the corresponding sequences in the heavy chain-encoding plasmid. Full length hIgG1 GLP1.1, GLP1.2, BLP1.1, and BLP1.2 antibodies,” in which the modified heavy chains as described above were paired with a human IgG1 light chain, were produced recombinantly via transfection into Expi293 cells and purified using MabSelect Sure resin.

Binding of the modified versions of LP1 to huTfR was assessed using BLI as described in Example 2. The results, shown in FIG. 8 (lower left graph) demonstrate that BLP1.1 improved maximum binding (Rmax) to huTfR by about 8-fold relative to LP1 while also showing a substantial improvement in binding affinity (kD).

### Example 4: Internalization of CDRH3-Grafted Antibodies

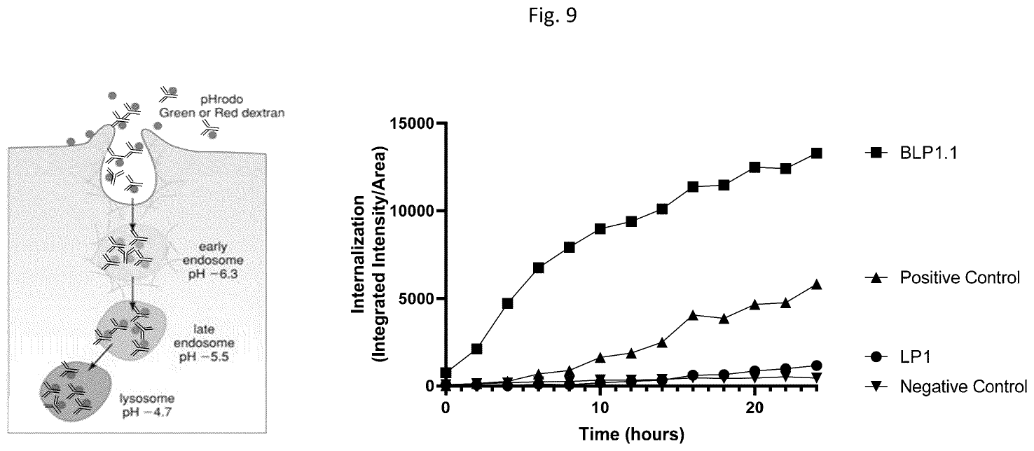

Assays were performed to measure the internalization of CDRH3-grafted antibodies. A diagram of the assay is provide in FIG. 9. Increased internalization results in increased red fluorescence signal. In these assays, target cells (hCMEC/D3) were seeded in the inner wells of a black wall clear bottom 96-well plate (#3904, Corning) at a concentration of 2.5*10\hat{ }4 cells/well in 100 μl of EBM2 media. Cells were allowed to adhere for 24 hours (hrs) at 37° C. and 5% CO2. On day 2, test antibodies were diluted to 10 μg/mL in EBM2 media, and then 110 μL of diluted antibody was added to 110 μL of 10 μg/mL pHrodo-conjugated anti-huIgG antibody also diluted in EBM2 media. This reagent was generated from Goat (Fab2) anti-human IgG (Jackson 109-006-008) conjugated using a pHrodo iFL Red Microscale Protein Labeling Kit (P36014) kit from ThermoFisher. The antibody/pHrodo mix was allowed to incubate for 37° C. for 15 minutes to allow for binding of the secondary Ab to the test antibody. After equilibration, 50 μL of media was removed from each of the hCMEC/D3-containing wells, and 50 μL of the antibody/pHrodo mix was added. Additionally, control wells were incubated with pHrodo-labelled secondary antibody alone to monitor background signal. The 96-well plate was then transferred into an Incucyte S3 Live-cell imager (Sartorius), and the red fluorescence signal was measured every 1 hr for 24 hrs. Data was analyzed by the Incucyte software, fluorescent signal at each time point was normalized against cell area and plotted in PRISM 9 to generate time-course figures. The results are shown in FIG. 9 and demonstrate that BLP1.1 antibody showed increased internalization compared to a positive control antibody, the parental CDRH3-grafted antibody (LP1), and an isotype control.

### Example 5: Cross-Reactivity and Retention of huFcRn Binding of BLP1.1

Assays were performed in order to measure BLP1.1 binding. In these assays BLP1.1 and control antibodies were diluted to 5.0 g/mL and loaded onto Protein L tips (GatorBio, Cat. No. 160007) for two minutes at 600 rpm, followed by a wash step for 60 seconds at 1000 rpm in running buffer. Antibody-loaded tips were associated to 1.5 μM of antigen (Human FcRn/FCGRT&B2M Heterodimer Protein, His Tag, Acro Biosystems, Cat. No. FCN-H52W7; other antigens were cyno TfR, murine TfR, and TfR apical domain) solution, for three minutes at 1000 rpm, followed by 10 minutes of dissociation in running buffer. Tip regeneration was performed by dipping tips twice into 1/200 (v/v) phosphoric acid solution, for 15 seconds each, at 1000 rpm, with 15 seconds wash step in running buffer, after each regeneration injection. Antibody and antigen solutions were diluted in running buffer. A sample well with a positive control (anti-TfR huIgG1) and a sample well with a negative control (isotype control huIgG1) were included in each loading column to monitor instrument variability.

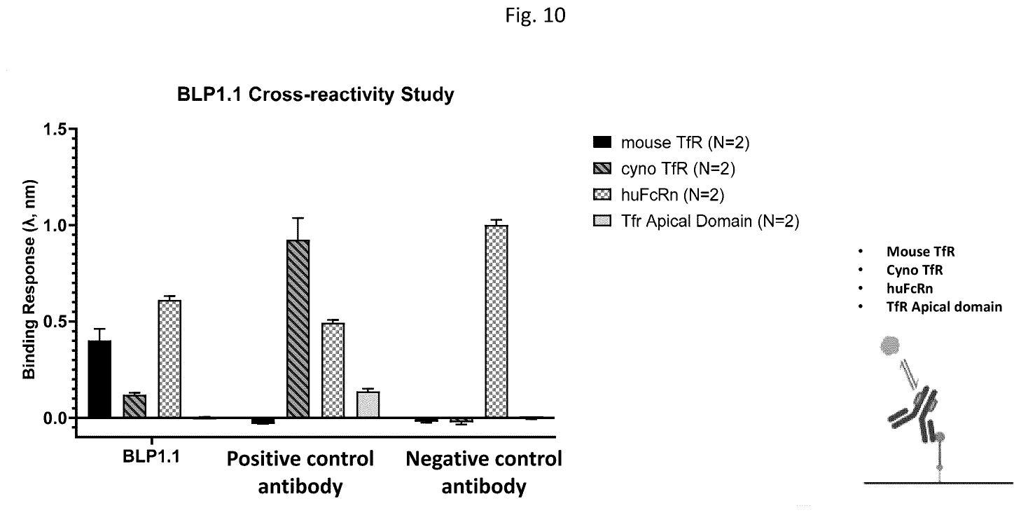

Raw data from active surfaces (antibody present) was double-referenced with reference tips (antibody absent) and buffer injections, over active surfaces. Report point data (shift, nm) were taken at 170 seconds from referenced sensogram data and compared with data collected with a negative control (antigen). Data was collected and analyzed with the GatorBio Software (version 2.0.3.0312). The results are shown in FIG. 10 and demonstrate that BLP1.1 is cross reactive with cynomolgus TfR and murine TfR and retains FcRn binding. The results also demonstrate that BLP1.1 does not bind at the TfR apical domain.

### Example 6: Effects of BLP1.1 on TfR

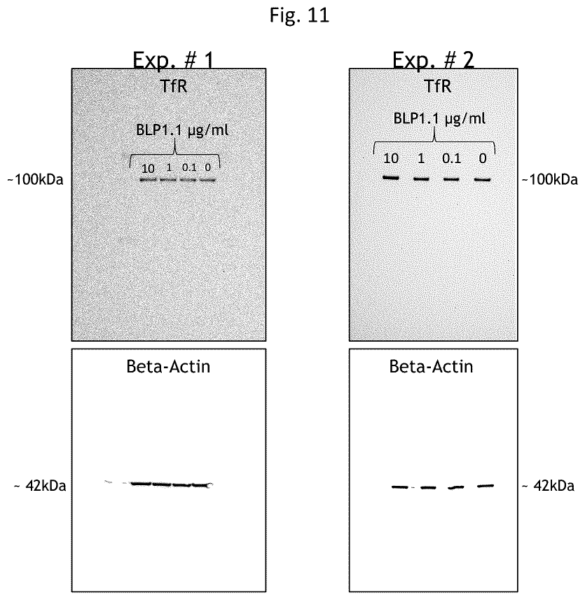

To assess effects of BLP1.1 on TfR expression, HCMEC/D3 cells, a cell line derived from human brain endothelial cells, were seeded in a 12-well plate at 3×10\hat{ }5 cells per well. On the next day (˜16 hr), the cells were incubated with three different concentrations of BLP1.1 antibody (Ab) (0.1, 1 and 10 μg/mL) for 24 hr at 37° C. Cells were then washed twice with ice-cold PBS and lysed in radioimmunoprecipitation assay (RIPA) buffer (Sigma-Aldrich R0278) containing a cocktail of proteases inhibitors. After 30 min shaking on a nutator at 4° C., cells lysates were spun at max speed (15000 rpm) in a benchtop centrifuge for 20 min at 4° C. The supernatants were then transferred into new tubes and mixed with sampling buffer, followed by 5 min incubation on a heat block (95° C.). Next, samples were run on a protein gel and transferred onto a nitrocellulose membrane, which was then blocked with blocking buffer (TBST+5% milk powder), and probed with 1 μg/mL of anti-TfR Ab for 24 hr at 4° C. After 4 washes with PBST, the membrane was probed with an HRP-conjugated goat anti-human IgG1 Ab (1:3000 dilution) for 1 hr at room temperature, followed by 4 washes with PBST. The membrane was then developed with Supersignal West Pico reagent, imaged and analyzed using an iBright instrument. Next, the membrane was incubated with stripping buffer to remove the bound Abs, before being probed with anti-human b-actin for quantification purposes. The results, shown in FIG. 11, revealed no differences in amount of total TfR protein between different treatments, indicating that BPL1.1 does not affect the TfR turnover in HCMEC/D3 cells.

The effect of BLP1.1 on TfR expression in vivo was also examined. In these assays, a group of mice (N=24) were inoculated with 10 mg/kg of either BLP1.1 or human IgG1 isotype antibodies (12 mice in each group). At 1-, 3- and 7-days post treatment, mice were anesthetized and underwent cardiac perfusion with 30 mLs of PBS. Brain tissues were then harvested and underwent vessel depletion to separate the vessels portion from brain parenchyma. Isolated vessels were subsequently lysed and used in western blot to quantify total TfR protein. Similar to the results from the in vitro assay, no differences in the amount of TfR protein between the two groups at any time-points were confirmed. These results further confirm that BLP1.1 does not alter TfR turnover in target cells. Additionally, cell-surface expression of TfR was not affected upon BLP1.1 treatment, as assessed by FACS.

### Example 7: Melting Temperature (Tm) Determination Via Differential Scanning Fluorimetry

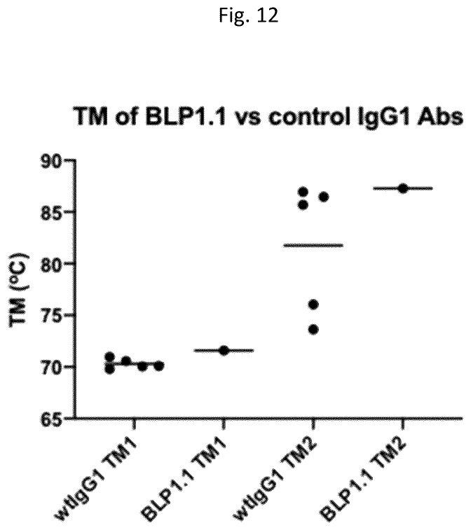

Differential scanning fluorimetry (DSF) analysis measures protein thermal stability by performing temperature scan from 25-95° C. at a rate of 1° C. per minute using Protein Thermal Shift Dye Kit and software from Thermo Fisher Scientific. As the temperature scan increases, the protein begins to unfold and expose hydrophobic patches; the fluorescent Rox dye binds to the patches of the unfolding protein and can be measured by qPCR (Quant Studio 3 from Applied Biosystems by Thermo Fisher Scientific). The different baseline between the beginning and ending is the differential melting temperature, ΔTm. The higher Tm values suggest a more stable native protein. The results of DSF analysis of BLP1.1 are shown in FIG. 12. TM1 was measured at 71.6° C., and TM2 was measured at 87.3° C. The results demonstrate that BLP1.1 has an equivalent melting temp (TM) to WT IgG1 Abs.

### Example 8: In Vivo PK Study in Vessel-Depleted Brain Tissue

The ability of CDRH3-grafted antibodies to enter brain tissues can be measured via IV injection and harvesting of brain tissues in a murine model. In such assays, mice are IV injected with 10 mg/kg of either CDRH3-grafted antibody or an isotype control (N=12 per antibody). Serum is collected 1 hr, 4 hr, 24 hr, 72 hr and 168 hr post-injection to monitor the systemic PK of the antibodies. Mice (N=4) are euthanized at 24 hr, 72 hr and 168 hr post-injection, and undergo cardiac perfusion with ice-cold PBS. Brains are harvested and separated into vessel and parenchymal fractions, as is commonly performed in the field via centrifugation. The parenchymal fraction is lysed, and total protein concentration determined using a Pierce™ BCA Protein Assay Kit (ThermoScientific #23225).

The amount of CDRH3-grafted antibody in sera or vessel depleted brain samples is quantified using a custom Meso Scale Discovery (MSD) assay. Briefly, MSD plates are coated with goat anti-human Fab specific IgG (Jackson Immuno #109-005-097) at 1 μg/mL in PBS overnight at 4° C. After coating, the plates are blocked with 1% BSA in PBS for 1 hr at room temperature (RT) with shaking at 500 rpm. Meanwhile, serum samples are serially diluted to a final dilution of 1:10,000 in assay buffer (PBS+1% BSA+0.05% Tween20), and the lysed brain tissue diluted 1:10 in assay buffer. The blocked plate is then washed 3× with assay buffer, and the diluted serum, brain lysate, and control IgG dilution series are added and incubated for 1 hr at RT with shaking at 500 rpm. Following primary incubation, the plate is again washed 3× with assay buffer, and detection antibody (anti-human sulfo-Tag, 25 μL of 1 μg/mL in assay buffer per well) is added and incubated for 1 hr at RT with shaking at 500 rpm. After a final series of 3 washes in assay buffer, 150 μL of 1× Read buffer is added per well, and the plates are read on the Sector Imager (MSD S600). The MSD software then uses the standard curve included on each plate to calculate a human IgG level for each sample.

BLP1.1 demonstrated instability, rapid clearance in plasma and brain (CNS tissue), and therefore did not improve trafficking to brain parenchyma.

Taken together, these assays demonstrate that other antibodies grafted with a CDRH3 BBB-binding domain and that have better stability, would be expected to increase accumulation in the brain as compared to non-grafted antibodies.

The present description is not to be limited in scope by the specific aspects described herein. Indeed, various modifications of the present disclosure, in addition to those described, will be apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Additional aspects are within the following claims.

1. A polypeptide comprising an engineered constant domain, wherein the engineered constant domain comprises a heterologous insertion in a constant domain, and wherein the heterologous insertion comprises a heavy chain complementary determining region 3 (CDRH3) or a target-binding fragment thereof.

2. The polypeptide of claim 1, wherein the constant domain comprises a CH3 domain, a CH2 domain, a CH1 domain, or a CL domain.

3-6. (canceled)

7. The polypeptide of claim 1, wherein the CDRH3 or the target-binding fragment thereof specifically binds to a blood brain barrier (BBB) target.

8-10. (canceled)

11. A fusion protein comprising an engineered constant domain and a heterologous polypeptide, wherein the engineered constant domain comprises a heterologous insertion in a constant domain, and wherein the heterologous insertion comprises a CDRH3 or a target-binding fragment thereof.

12-16. (canceled)

17. The fusion protein of claim 11, wherein the CDRH3 or target-binding fragment thereof specifically binds to a BBB target.

18-25. (canceled)

26. The fusion protein of claim 11, wherein the heterologous polypeptide comprises an antibody or antigen-binding fragment thereof, a growth factor, a decoy receptor, or an enzyme or a catalytically active fragment thereof.

27-29. (canceled)

30. An antigen-binding protein comprising an engineered constant domain and an antigen-binding domain, wherein the engineered constant domain comprises a heterologous insertion in a constant domain, and wherein (i) the heterologous insertion comprises a CDRH3 or a target-binding a fragment thereof or (ii) the heterologous insertion specifically binds to a blood brain barrier target and the antigen-binding domain binds to a different target than the heterologous insertion.

31-42. (canceled)

43. The polypeptide of claim 1, wherein the constant domain is an IgG constant domain.

44-51. (canceled)

52. The polypeptide of claim 1, wherein the heterologous insertion binds to transferrin receptor, Basigin, Glucose Transporter Type 1 (Glut1), CD98, MfsD2a, LRP1, LRP8, or IGF1R.

53-66. (canceled)

67. The polypeptide of claim 1, wherein the heterologous insertion comprises the amino acid sequence
(SEQ ID NO: 11)
NVYAMTRNWWSDVY,
(SEQ ID NO: 12)
NVWHDLVWSVSTTDVY,
(SEQ ID NO: 13)
NVYARPRPDNLNWSDVY,
(SEQ ID NO: 14)
HVETPYDSPELNWWDVY,
[[or]]
(SEQ ID NO: 15)
NVYELVEDTSAYEIGVDVY,
(SEQ ID NO: 61)
MLLRSDWYFDVW,
(SEQ ID NO: 16)
TGWGFDYW,
(SEQ ID NO: 17)
TRGATALDYW,
(SEQ ID NO: 18)
NVYAMTRNWWSDVY,
(SEQ ID NO: 19)
NVWHDLVWSVSTTDVY,
(SEQ ID NO: 20)
NVYARPRPDNLNWSDVY,
(SEQ ID NO: 21)
HVETPYDSPELNWWDVY,
(SEQ ID NO: 22)
NVYELVEDTSAYEIGVDVY,
(SEQ ID NO: 23)
ARGLSGNYV,
(SEQ ID NO: 24)
ARGLSGNFV,
(SEQ ID NO: 25)
GLSGNYVMDY,
(SEQ ID NO: 26)
GLSGNYVVDY,
(SEQ ID NO: 27)
GLSGNFVMDF
(SEQ ID NO: 28)
ARGTRA
(SEQ ID NO: 29)
GTRAYHY,
(SEQ ID NO: 30)
GTYRAYHF
(SEQ ID NO: 31)
ARYGYGNPATRY,
(SEQ ID NO: 32)
YGYGNPATRYFDV,
(SEQ ID NO: 33)
TRGGYDSRAWF,
(SEQ ID NO: 34)
GGYDSRAWFAY,
(SEQ ID NO: 35)
GGYDSRAWFAH,
(SEQ ID NO: 36)
ARQGALYDGYYRGA,
(SEQ ID NO: 37)
QGALYDGYYRGAMDY,
(SEQ ID NO: 38)
ARRGYGYDGEF,
(SEQ ID NO: 39)
RGGYGYDGEFAY,
(SEQ ID NO: 40)
GLSGN(Y/F)V(M/V)D(Y/F),
(SEQ ID NO: 41)
GTRAYH(Y/F),
(SEQ ID NO: 42)
(G/Q/R)G(Y/A/G)(D/L/Y)(S/Y/G)(R/D/Y)
(A/G/D)(W/Y/G)(F/Y/E)(R/F/-)(G/-)
(A/-)(M/-)(A/D)(Y/H),
(SEQ ID NO: 43)
VRLRGYFDY,
(SEQ ID NO: 44)
ARRDGSYYPYYWYFDL,
(SEQ ID NO: 45)
ARTLINYSDYADYVMDA,
(SEQ ID NO: 46)
ARSLINYRNYGDYVMDA,
(SEQ ID NO: 47)
ARNGVYHNYWYFDF,
or
(SEQ ID NO: 48)
AKEGGWFLRIYGMDY.

68-80. (canceled)

81. The antigen-binding protein of claim 30, wherein the antigen-binding protein is an antibody.

82. The antigen-binding protein of claim 30, wherein the antigen-binging proteins is an antigen-binding fragment of an antibody.

83-92. (canceled)

93. The antigen-binding protein of claim 30, wherein the antigen-binding protein comprises the amino acid sequence of SEQ ID NO:7.

94. The polypeptide of claim 1, further comprising a detectable label.

95. A library comprising a plurality of polypeptides of claim 1.

96. A polynucleotide encoding the polypeptide of claim 1.

97. An expression system comprising the polynucleotide of claim 96, optionally wherein the expression system is a host cell.

98. A polynucleotide comprising a nucleic acid sequence encoding a polypeptide comprising an engineered constant domain, wherein the engineered constant domain comprises a heterologous insertion in a constant domain, wherein the heterologous insertion (i) comprises a CDRH3 or a target-binding fragment thereof and/or (ii) specifically binds to a BBB target.

99-100. (canceled)

101. A pharmaceutical composition comprising the polypeptide of claim 1 and a pharmaceutically acceptable carrier.

102. (canceled)

103. A method for making a polynucleotide encoding the polypeptide of claim 1, the method comprising (a) providing a first nucleic acid sequence encoding the constant domain and (b) inserting a heterologous nucleic acid sequence into the region of the first nucleic acid sequence encoding the constant domain to produce the polynucleotide, wherein the heterologous nucleic acid sequence encodes a CDRH3 or a target-binding fragment thereof.

104-108. (canceled)

109. A method of producing an antigen-binding protein comprising culturing the host cell of claim 97, optionally wherein the method further comprises recovering the antigen-binding protein from the culture.

110. A method for adding a binding specificity to a protein comprising a constant domain, the method comprising making the polypeptide of claim 1 by (a) providing a first nucleic acid sequence encoding a protein comprising a constant domain, (b) inserting a heterologous nucleic acid sequence into the first nucleic acid sequence to produce a modified polynucleotide, wherein the heterologous nucleic acid sequence encodes a CDRH3 or a target-binding fragment thereof, and (c) expressing the modified polynucleotide in an expression system.

111. A method for increasing the ability of a protein comprising a constant domain to cross a blood brain barrier, the method comprising (i) producing the polynucleotide of claim 98 by (a) providing a first nucleic acid sequence encoding a protein comprising the constant domain [,] and (b) inserting a heterologous nucleic acid sequence into the region of the first nucleic acid sequence encoding the constant domain to produce a modified polynucleotide, wherein the heterologous nucleic acid sequence encodes an amino acid sequence that binds to a blood brain barrier target and (ii) expressing the modified nucleic acid sequence in an expression system.

112-117. (canceled)

118. A method for identifying a protein with the ability to cross a blood brain barrier, the method comprising (i) creating a library comprising a plurality of polynucleotides of claim 96, (ii) expressing the polypeptides encoded by the library, and (iii) screening the polypeptides for the ability to bind to a BBB target and/or cross the BBB.

119. A method for producing a protein with the ability to cross a BBB,
the method comprising (i) screening a library of proteins containing CDRH3 domains for the ability to bind a blood brain barrier target, (ii) selecting a protein from the library of proteins and identifying the CDRH3 domain of the selected protein, (iii) inserting a nucleic acid sequence encoding the CDRH3 domain or a target-binding fragment thereof of the selected protein into a nucleic acid sequence encoding a protein comprising a constant domain to produce a modified polynucleotide, wherein the insertion is within the region of the nucleic acid encoding the constant domain to produce a polynucleotide encoding the polypeptide of claim 1, and (iv) expressing the modified polynucleotide in a host cell.

120-121. (canceled)

122. A method for producing a protein with the ability to cross a BBB, the method comprising (i) inserting a nucleic acid sequence encoding a CDRH3 or a target-binding fragment thereof into a nucleic acid sequence encoding a protein comprising a constant domain to produce a polynucleotide, wherein the insertion is within the region of the nucleic acid encoding the constant domain, (ii) expressing the polynucleotide to produce the polypeptide of claim 1, and (iii) screening the polypeptide for the ability to bind a blood brain barrier target and/or cross the blood brain barrier, optionally wherein the CDRH3 or target-binding fragment thereof is derived from an antibody that binds the BBB target.

123-124. (canceled)

125. A method for engineering a protein capable of crossing the BBB, the method comprising (i) producing the polypeptide of claim 1 by (a) providing a first nucleic acid encoding a protein, wherein the protein comprises a constant domain, (b) inserting a heterologous nucleic acid encoding a CDRH3 domain or target-binding fragment thereof into the portion of the first nucleic acid that encodes the constant domain to produce a modified nucleic acid encoding the polypeptide of claim 1, and (c) expressing the modified nucleic acid in an expression system to produce the polypeptide of claim 1, and (ii) screening the polypeptide of claim 1 for binding to a BBB target and/or for crossing the BBB.

126-129. (canceled)

130. A method of detecting an antigen in the central nervous system of a subject, the method comprising administering the antigen-binding protein of claim 30 to the subject and detecting the binding of the antigen-binding protein to the antigen in the central nervous system, optionally wherein the antigen is in the brain.

131. A method of transporting a polypeptide, fusion protein, antigen-binding protein across a BBB in a subject, the method comprising administering to the subject the polypeptide, fusion protein, or antigen-binding protein of claim 1.

132. A method of treating a neurological disease or disorder in a subject comprising administering the polypeptide, fusion protein, or antigen-binding protein of claim 1 to the subject.

133-135. (canceled)
